(19) United States
(12) Patent Application Publication
(10) Pub. No.: US 2025/0114441 A1
(43) Pub. Date: Apr. 10, 2025

(54) POLYMERIC VACCINE FOR OPIOID ADDICTION

(71) Applicant: The Johns Hopkins University, Baltimore, MD (US)

(72) Inventors: David Scott Wilson, Baltimore, MD (US); Sandeep Kumar, Baltimore, MD (US)

(21) Appl. No.: 18/912,388

(22) Filed: Oct. 10, 2024

Related U.S. Application Data
(60) Provisional application No. 63/543,445, filed on Oct. 10, 2023.

(51) Int. Cl. A61K 39/00; A61K 39/385; A61K 47/64; A61P 25/36

(52) U.S. Cl. CPC A61K 39/0013; A61K 39/385; A61K 47/646; A61P 25/36; A61K 2039/6081; A61K 2039/627

(57) ABSTRACT

Disclosed herein are immune activating conjugates with immunogenic carrier proteins coupled to hapten and immune cell receptor agonist-containing polymers. The polymers promote uptake by B cells, enabling intracellular immune cell receptor activation and formation of long-lived antibody-secreting cells. In some aspects, disclosed herein in is a conjugate comprising an immunogenic carrier protein coupled to a polymer, wherein the polymer includes: i) a first monomeric unit comprising a first monomer coupled to a hapten, and ii) a second monomeric unit comprising a second monomer coupled to an immune cell receptor agonist.

### CROSS-REFERENCE TO RELATED APPLICATIONS

This application claims the benefit of priority under 35 U.S.C. § 119(e) to U.S. Provisional Application No. 63/543,445, filed Oct. 10, 2023. The content of the prior application is considered part of and is hereby incorporated by reference in its entirety.

### BACKGROUND OF THE INVENTION

### Field of the Invention

The present disclosure relates generally to vaccines and more specifically to polymer-protein conjugates that stimulate a B cell response against specific haptens.

### Background Information

Opioid addiction and overdose related deaths have increased at an alarming rate. The number of patients suffering from opioid abuse disorders increased 439% from 2010 to 2016, while the number of opioid-related deaths increased by 646% over that same time period (CDC report). This exponential rise in the death rate has been attributed to the fact that those who use elicit opioids, such are heroin and morphine, are increasingly likely to unknowingly consume powerful synthetic opioids, such as fentanyl, which are orders of magnitude more deadly. In response to this mounting national epidemic, the National Institutes for Health has increased its efforts toward the development of novel, more effective treatment strategies for opioid use disorders with a focus on synthetic opioids. One promising strategy to prevent opioid overdose and enhance recovery is the use of synthetic opioid-targeted vaccines to elicit antibodies that selectively bind to the drugs in serum to form immunocomplexes that are too large to enter the brain and peripheral organs. However, repeatedly vaccinating any patient, much less a drug addict, over many years is a challenge. Therefore, there is an urgent need of developing a platform that can generate robust, durable a α-opioid responses that persist for years.

### SUMMARY OF THE INVENTION

The present disclosure is based on the seminal discovery of a polymeric conjugate that induces durable hapten (e.g., α-opioid) responses by crosslinking B cell receptors (BCRs) to activate B cells. Leveraging this discovery, in some embodiments, the present disclosure provides a conjugate that includes an immunogenic carrier protein coupled to a polymer, wherein the polymer includes: i) a first monomeric unit with a first monomer coupled to a hapten, and ii) a second monomeric unit with a second monomer coupled to an immune cell receptor agonist.

In some aspects, the polymer also includes a third monomeric unit comprising a third monomer. In particular aspects, the third monomeric unit does not include a hapten, drug, or biomolecule. In certain aspects, the third monomeric unit has a molecular mass of less than about 250 Da, less than about 225 Da, less than about 200 Da, less than about 175 Da, less than about 150 Da, less than about 125 Da, or less than about 100 Da. In a particular aspect, the third monomeric unit is an acrylate, an acrylamide, a methacrylate, a methacrylamide, or a vinyl group. In another aspect, the third monomeric unit is hydroxyethyl methacrylamide. In particular aspects, the first monomer and the second monomer are each independently an acrylate, an acrylamide, a methacrylate, a methacrylamide, or a vinyl group. In certain aspects, the first monomer is identical to the second monomer. In further aspects, the first, second, and third monomer are identical.

In one aspect, the first monomeric unit and the second monomeric unit are between about 1% and 50%, between about 1% and 5%, between about 1% and 10%, between about 1% and 15%, between about 1% and 20%, between about 1% and 25%, between about 1% and 30%, between about 1% and 40%, between about 2% and 5%, between about 2% and 10%, between about 2% and 15%, between about 2% and 20%, between about 2% and 25%, between about 2% and 30%, between about 2% and 40%, between about 2% and 50%, between about 5% and 10%, between about 5% and 15%, between about 5% and 20%, between about 5% and 25%, between about 5% and 30%, between about 5% and 40%, between about 5% and 50%, between about 10% and 20%, between about 10% and 30%, between about 10% and 40%, between about 10% and 50%, between about 15% and 25%, between about 15% and 30%, between about 15% and 40%, between about 15% and 50%, between about 20% and 30%, between about 20% and 40%, between about 20% and 50%, between about 25% and 40%, between about 25% and 50%, between about 30% and 40%, or between about 30% and 50% of monomeric units of the polymer.

In another aspect, the first monomeric unit is between about 0.5% and 2.5%, between about 0.5% and 5%, between about 0.5% and 7.5%, between about 0.5% and 10%, between about 0.5% and 15%, between about 0.5% and 20%, between about 0.5% and 25%, between about 1% and 2.5%, between about 1% and 5%, between about 1% and 7.5%, between about 1% and 10%, between about 1% and 15%, between about 1% and 20%, between about 1% and 25%, between about 2.5% and 5%, between about 2.5% and 7.5%, between about 2.5% and 10%, between about 2.5% and 15%, between about 2.5% and 20%, between about 2.5% and 25%, between about 5% and 7.5%, between about 5% and 10%, between about 5% and 15%, between about 5% and 20%, between about 5% and 25%, between about 7.5% and 10%, between about 7.5% and 15%, between about 7.5% and 20%, between about 7.5% and 25%, between about 10% and 15%, between about 10% and 20%, between about 10% and 25%, or between about 15% and 25% of monomeric units of the polymer. In a particular aspect, first monomeric unit is about 9% of the monomeric units of the polymer.

In a further aspect, the second monomeric unit is between about 0.5% and 2.5%, between about 0.5% and 5%, between about 0.5% and 7.5%, between about 0.5% and 10%, between about 0.5% and 15%, between about 0.5% and 20%, between about 0.5% and 25%, between about 1% and 2.5%, between about 1% and 5%, between about 1% and 7.5%, between about 1% and 10%, between about 1% and 15%, between about 1% and 20%, between about 1% and 25%, between about 2.5% and 5%, between about 2.5% and 7.5%, between about 2.5% and 10%, between about 2.5% and 15%, between about 2.5% and 20%, between about 2.5% and 25%, between about 5% and 7.5%, between about 5% and 10%, between about 5% and 15%, between about 5% and 20%, between about 5% and 25%, between about 7.5% and 10%, between about 7.5% and 15%, between about 7.5% and 20%, between about 7.5% and 25%, between about 10% and 15%, between about 10% and 20%, between about 10% and 25%, or between about 15% and 25% of monomeric units of the polymer. In another aspect, the second monomeric unit is about 15% of the monomeric units of the polymer.

In certain aspects, the first, second, and third monomeric units are between about 50% and 80%, between about 50% and 90%, between about 50% and 100%, between about 60% and 80%, between about 60% and 90%, between about 60% and 100%, between about 70% and 80%, between about 70% and 90%, between about 70% and 100%, between about 80% and 90%, between about 80% and 100%, between about 90% and 100%, between about 95% and 100%, about 100%, or 100% of the monomeric units of the polymer.

In one aspect, the polymer is a random copolymer. In another aspect, the first monomer and the second monomer are non-peptidic.

In some aspects, the polymer has a molecular weight of between about 0.1 to 200 kDa, between about 0.1 to 0.5 kDa, between about 0.1 to 1 kDa, between about 0.5 to 5 kDa, between about 0.5 to 10 kDa, between about 1 and 5 kDa, between about 1 and 10 kDa, between about 1 and 20 kDa, between about 1 and 30 kDa, between about 1 and 40 kDa, between about 1 to 50 kDa, between about 2 and 5 kDa, between about 2 and 10 kDa, between about 2 and 20 kDa, between about 2 and 30 kDa, between about 2 and 40 kDa, between about 2 and 50 kDa, between about 5 and 10 kDa, between about 5 and 20 kDa, between about 5 and 30 kDa, between about 5 and 40 kDa, between about 5 and 50 kDa, between about 5 and 100 kDa, between about 10 and 20 kDa, between about 10 and 30 kDa, between about 10 and 40 kDa, between about 10 and 50 kDa, between about 10 and 100 kDa, between about 10 and 200 kDa, between about 20 and 30 kDa, between about 20 and 40 kDa, between about 20 and 50 kDa, between about 20 and 100 kDa, between about 20 and 150 kDa, between about 20 and 200 kDa, between about 30 and 40 kDa, between about 30 and 50 kDa, between about 30 and 100 kDa, between about 30 and 150 kDa, between about 30 and 200 kDa, between about 50 and 100 kDa, between about 50 and 150 kDa, between about 50 and 200 kDa, between about 100 and 150 kDa, or between about 100 and 200 kDa. In a particular aspect, the polymer has a molecular weight of about 24 kDa.

In certain aspects, the polymer has between about 10 and 5000 monomeric units.

In some aspects, the hapten is an opioid. In certain aspects, the opioid is fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, or pharmacologically acceptable salt or solvate thereof. In a particular aspect, the opioid is fentanyl, a derivative or analog thereof, a precursor thereof, or pharmacologically acceptable salt or solvate thereof. In some aspects, the fentanyl analog is acetyl fentanyl, parafluorofentanyl, furanyl fentanyl, or carfentanil. In a specific aspect, the opioid is fentanyl. In some aspects, the hapten is irreversibly coupled to the first monomer.

In another aspect, the hapten is a chemical warfare agent. In some aspects, the chemical warfare agent is agent orange, 3-quinuclinidyl benzilate, VX gas, VR gas, HN1, HN2, or Tabun.

In a further aspect, the hapten is nicotine, cocaine, or MDMA.

In a further aspect, the hapten is a peptide or sugar derived from an infectious agent.

In certain aspects, the immune cell receptor agonist is an agonist of an intracellular receptor. In a particular aspect, the immune cell receptor agonist is a toll-like receptor agonist. In a further aspect, the immune cell receptor agonist is a toll-like receptor 7 (TLR7) agonist, a toll-like receptor 8 agonist (TLR8), or a TLR7/TLR8 agonist. In another aspect, the immune cell receptor agonist is a TLR7 agonist.

In one aspect, the TLR7 agonist is imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, S-34240, SBT-6050, SBT-6290, ZM-TLR8, VX-001, MBS-8, APR-002, or a combination thereof. In another aspect, the TLR7 agonist is an imidazoquinoline. In a further aspect, the TLR7 agonist is

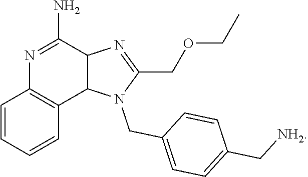 [SMILES: CCOCC1=NC2C(N)=Nc3ccccc3C2N1Cc1ccc(CN)cc1]

In some aspects, the immune cell receptor agonist is coupled to the second monomer by a cleavable linker. In certain aspects, the cleavable linker includes a carbamate. In some aspects, the immunogenic carrier protein is a pathogenic protein. In a particular aspect, the immunogenic carrier protein is a bacterial protein. In one aspect, the immunogenic carrier protein is a tetanus protein. In another aspect, the immunogenic carrier protein is a tetanus toxin or other immunogenic protein. In one aspect, the immunogenic carrier protein is a diphtheria toxin. In another aspect, the immunogenic carrier protein is a genetically detoxified diphtheria toxin. In a further aspect, the immunogenic carrier protein is a diphtheria toxin. In a particular aspect, the immunogenic carrier protein is CRM197.

In some aspects, the immunogenic carrier protein coupled to the polymer by a cleavable linker. In one such aspect, the cleavable linker is a self-immolating linker. In another aspect, cleavage of the cleavable linker releases the immunogenic carrier protein in an unmodified form.

In some aspects, the conjugate is water-soluble. In further aspects, the conjugate is non-toxic. In additional aspects, the conjugate is configured to internalize within a B cell. In particular aspects, the B cell is an opioid-specific B cell.

In a further embodiment, the present disclosure provides a pharmaceutical composition that includes the conjugate of the present disclosure and a pharmaceutically acceptable excipient.

In another embodiment, the present disclosure provides a method of treating addiction in a subject in need thereof that includes administering the conjugate or the composition of the present disclosure to the subject, thereby treating the addiction in the subject. In some aspects, the addiction is opioid addiction.

In a further embodiment, the present disclosure provides a method of preventing drug overdose in a subject in need thereof including administering the conjugate or the composition of the present disclosure to the subject, thereby preventing the overdose in the subject. In some embodiments, the overdose is an opioid overdose.

In some embodiments, the present disclosure provides a vaccine composition including a conjugate including an immunogenic carrier protein coupled to a polymer, wherein the polymer includes: a first monomeric unit including a first monomer coupled to a hapten, a second monomeric unit including a second monomer coupled to an immune cell receptor agonist, and a third monomeric unit comprising a third monomer.

In some aspects, the hapten is an opioid selected from the group consisting of fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, or pharmacologically acceptable salt or solvate thereof.

In some aspects, the immune cell receptor agonist is a toll-like receptor (TLR) agonist selected from the group consisting of imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, S-34240, SBT-6050, SBT-6290, ZM-TLR8, VX-001, MBS-8, APR-002, imidazoquinoline,

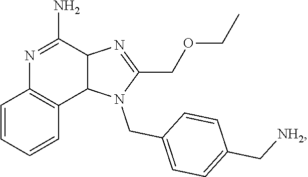 [SMILES: CCOCC1=NC2C(N)=Nc3ccccc3C2N1Cc1ccc(CN)cc1]

and fragment thereof.

In some aspects, the immunogenic carrier protein is selected from the group consisting of tetanus toxins, diphtheria toxins, related proteins such as CRM197, genetically toxoided variant of diphtheria toxin, meningococcal outer membrane protein complex (OMPC), H. influenzae protein D (HiD), Keyhole Limpet Hemocyanin (KLH), bovine serum albumin (BSA), ovalbumin (OVA), and recombinant P. aeruginosa exoprotein.

In some embodiments, the present disclosure provides a vaccine composition comprising a conjugate comprising ovalbumin coupled to a polymer, wherein the polymer includes: a first monomeric unit comprising a first monomer coupled to fentanyl, a second monomeric unit including a second monomer coupled to a TLR7 agonist, a TLR8 agonist, or a TLR7/TLR8 agonist, and a third monomeric unit including a third monomer.

In some embodiments, the present disclosure provides a vaccine composition comprising a conjugate comprising CRM197 coupled to a polymer, wherein the polymer includes: a first monomeric unit including a first monomer coupled to fentanyl, a second monomeric unit comprising a second monomer coupled to a TLR7 agonist, a TLR8 agonist, or a TLR7/TLR8 agonist, and a third monomeric unit including a third monomer.

In some embodiments, the present disclosure provides a method of vaccinating a subject in need thereof against substance abuse including administering to the subject a vaccine composition including an immunogenic carrier protein coupled to a polymer, wherein the polymer includes: a first monomeric unit including a first monomer coupled to a hapten, a second monomeric unit including a second monomer coupled to an immune cell receptor agonist, and a third monomeric unit comprising a third monomer, thereby vaccinating the subject against substance abuse.

In some aspects, the immunogenic carrier protein is ovalbumin, the hapten is fentanyl, and the immune cell receptor agonist a TLR7 agonist, a TLR8 agonist, or a TLR7/TLR8 agonist.

In some aspects, the immunogenic carrier protein is CRM197, the hapten is fentanyl, and the immune cell receptor agonist a TLR7 agonist, a TLR8 agonist, or a TLR7/TLR8 agonist.

In some aspects, the immunogenic carrier protein is Keyhole Limpet Hemocyanin (KLH), the hapten is fentanyl, and the immune cell receptor agonist a TLR7 agonist, a TLR8 agonist, or TLR7/TLR8 agonist.

In some embodiments, the present disclosure provides a method of preventing drug overdose in a subject in need thereof including administering to the subject a conjugate including an immunogenic carrier protein coupled to a polymer, wherein the polymer includes: a first monomeric unit including a first monomer coupled to a hapten, and a second monomeric unit including a second monomer coupled to an immune cell receptor agonist, thereby preventing drug overdose in the subject.

In some aspects, the immunogenic carrier protein is ovalbumin, the hapten is fentanyl, and the immune cell receptor agonist a TLR7 agonist, a TLR8 agonist, or a TLR7/TLR8 agonist.

In some aspects, the immunogenic carrier protein is CRM197, the hapten is fentanyl, and the immune cell receptor agonist a TLR7 agonist, a TLR8 agonist, or a TLR7/TLR8 agonist.

In some aspects, the immunogenic carrier protein is Keyhole Limpet Hemocyanin (KLH), the hapten is fentanyl, and the immune cell receptor agonist a TLR7 agonist, a TLR8 agonist, or a TLR7/TLR8 agonist.

### BRIEF DESCRIPTION OF THE DRAWINGS

FIG. 1A-1E are illustrations that depict interactions between hapten-containing species and immune proteins or cells, and the design of a polymeric conjugate opioid vaccine. FIG. 1A is an illustration of an opioid-antibody complex. FIG. 1B is an illustration that depicts B cell activation upon B cell receptor (BCR) crosslinking and T cell stimulation. FIG. 1C is an illustration that depicts an opioid conjugate vaccine with an opioid coupled directly to a carrier protein. FIG. 1D is an illustration that depicts a conjugate of the present disclosure. FIG. 1E is an illustration that depicts B cell activation by a conjugate of the present disclosure.

FIG. 2 illustrates a chemical structure of a conjugate of the present disclosure that includes an immunogenic carrier protein coupled to a polymer that contains a hapten and an immune cell receptor agonist.

FIG. 3 illustrates a chemical structure of a conjugate of the present disclosure that includes an immunogenic carrier protein coupled to a polymer that contains a hapten.

FIG. 4A-4D illustrate an illustration that depicts a vaccination regiment and graphs that summarize immune activation following the vaccination regimen. FIG. 4A illustrates an illustration that depicts a 91-day vaccination and blood collection regimen in mice. FIG. 4B illustrates a graph that summarizes antibody titers in mice following the vaccination regimen outlined in FIG. 4A with various conjugates. FIG. 4C illustrates a graph that summarizes antibody titers in mice at days 14, 21, 63, and 90 of the vaccination regimen outlined in FIG. 4A with various conjugates. FIG. 4D illustrates a graph that summarizes activity indices in mice following vaccination regimens with various conjugates.

FIG. 5A-5B illustrate bar graphs of anti-fentanyl antibody titers in mice following subcutaneous vaccination with polymeric or monomeric fentanyl conjugates. FIG. 5A illustrates a bar graph of IgG titers in the mice. FIG. 5B illustrates a bar graph of IgA titers in the mice.

FIG. 6A-6C illustrate the steps of the process of T cell-dependent B cell activation. FIG. 6A illustrates pathogens initiate clustering of BCRs, FIG. 6B illustrates pathogen-activated B cells present antigens and activation signals to helper T cells, FIG. 6C illustrates LLACs and memory B cells as the result of B cell activation in the presence of T cell help.

FIG. 7 illustrates synthesis of p(Fent-co-TLR7).

FIG. 8 illustrates synthesis of TT-p(Fent-co-TLR7).

FIG. 9 illustrates the vaccination timeline.

FIG. 10 illustrates the timeline for α-fentanyl durability studies.

FIG. 11 illustrates the timeline for antinociceptive and lethal challenge experiments.

FIG. 12A-12G illustrate the performance of polymeric vaccine vs monomeric vaccine.

FIG. 12A illustrates vaccination schedule with CRM-pTLR-b-fent and CRM-mFent conjugate at two different doses 4 μg and 18 ug (of CRM and TLR 7 agonist weight equiv.) (FIG. 12B) illustrates anti-fentanyl serum IgG antibody level determined by ELISA on week 5. FIG. 12C illustrates anti-fentanyl serum IgA antibody level determined by ELISA on week 5. FIG. 12D illustrates anti-carfentanyl IgG antibody level determined by ELISA on week 5. FIG. 12E illustrates Avidity index. FIG. 12F illustrates long term anti-fentanyl IgG responses measured over 20 weeks. FIG. 12G illustrates the percentage maximum possible effect (% MPE). All statistical differences were determined by one-way ANOVA using Brown-Forsythe test where p<0.05 represent statistical significance with respect to all other treatment groups or as indicated.

FIG. 13A-13C illustrate the long-term anti-fentanyl IgG responses. FIG. 13A illustrates anti-fentanyl serum IgG antibody level determined by ELISA on week 5. FIG. 13B is anti-fentanyl serum IgA antibody level. FIG. 13C illustrates long term anti-fentanyl IgG responses measured over 13 weeks. All statistical differences were determined by one-way ANOVA using Brown-Forsythe test where p<0.05 represent statistical significance with respect to all other treatment groups or as indicated.

FIG. 14A-14E illustrate that block polymer induces better protection. FIG. 14A illustrates C57Bl/6 mice vaccinated with OVA-pTLR-b-fent (block polymer), OVA-pTLR-co-fent (random polymer) and OVA-pTLR polymer with one terminal fentanyl at 10 μg of OVA and TLR weight equiv. per mouse on day 0 and 21. FIG. 14B illustrates anti-fentanyl serum IgG antibody level determined by ELISA on week 5. FIG. 14C illustrates anti-fentanyl serum IgA antibody level determined by ELISA on week 5. FIG. 14D illustrates avidity index. FIG. 14E illustrates the percentage maximum possible effect (% MPE). All statistical differences were determined by one-way ANOVA using Brown-Forsythe test where p<0.05 represent statistical significance with respect to all other treatment groups or as indicated.

FIG. 15A-15E illustrate that Self immolative linker improve anti-fentanyl responses. FIG. 15A illustrates anti-fentanyl serum IgG antibody titer determined by ELISA on week 4. FIG. 15B illustrates anti-fentanyl serum IgA antibody titer determined by ELISA on week 4. FIG. 15C illustrates area under the curve for serum IgG. FIG. 15D illustrates area under the curve for serum IgA. FIG. 15E illustrates avidity index. All statistical differences were determined by one-way ANOVA using Brown-Forsythe test where p<0.05 represent statistical significance with respect to all other treatment groups or as indicated.

### DETAILED DESCRIPTION OF THE INVENTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

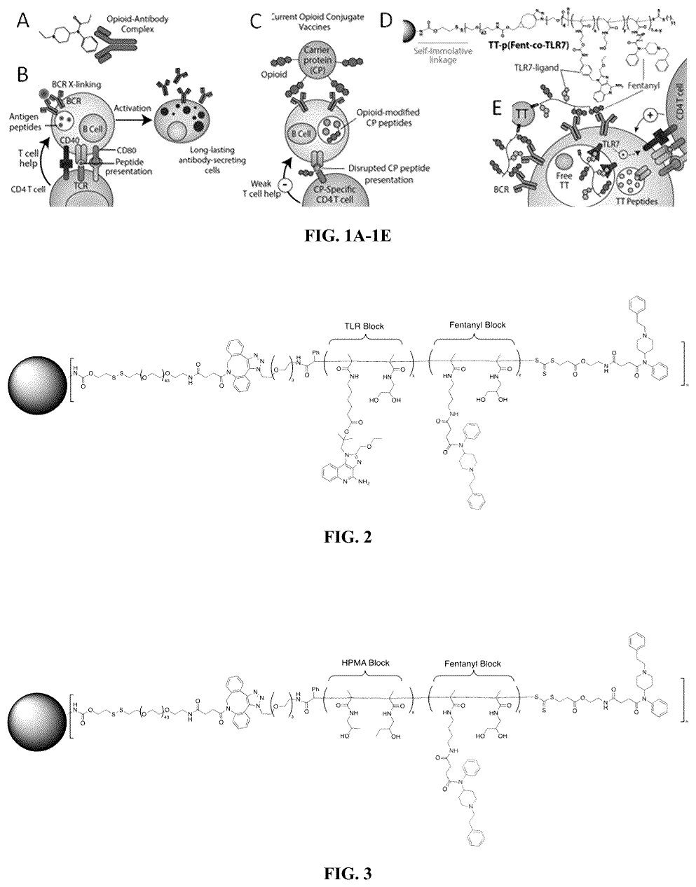

Opioid addiction is a national crisis, and opioid vaccines are a potentially lifesaving treatment. Described herein is vaccination to elicit opioid-specific antibodies (α-opioids) that bind to the drugs (FIG. 1A), preventing their passage into the organs and thus averting overdose. Effective opioid vaccines should be safe and generate durable α-opioid response, which are maintained by long-lived antibody-secreting cells (LLASCs). LLASCs are generated upon B cell receptor crosslinking and B cell recognition of signals from activated CD4⁺ T cells (i.e. T cell help)⁶ (FIG. 1B). Currently, to generate α-opioid responses, opioid monomers have been linked to carrier proteins to form conjugate vaccines that crosslink BCRs and activate T cell help through peptides derived from their carrier protein (FIG. 1C). However, to provoke responses in rodents, opioid conjugate vaccines are formulated with toxic experimental adjuvants that are not suitable for clinical use. Moreover, conjugating opioid monomers to carrier proteins can disrupt carrier protein peptide presentation to and recognition by carrier protein-specific T cells, greatly diminishing T cell help (FIG. 1C).

Described herein is demonstration of the efficacy of a polymeric opioid conjugate vaccine of the invention that induces durable α-opioid responses in the absence of toxicity. To maximize BCR crosslinking and activate B cells. In certain aspects, described herein is a water-soluble random copolymer, termed p(Fent-co-TLR7), composed of monomers decorated with either fentanyl-responsible for most opioid deaths—or a powerful (TLR7)-agonist (i.e., B cell-activating adjuvant). In some aspects, p(Fent-co-TLR7) was conjugated to the carrier protein tetanus toxin (TT) via a self-immolative linker that releases unmodified TT (FIG. 1D). In some embodiments, TT-p(Fent-co-TLR7) will elicit a robust, durable α-fentanyl response by efficiently crosslinking BCRs, activating B cell-TLR7 and maximizing the presentation of TT peptides (FIG. 1E). In some aspects, by targeting BCRs, which internalize when crosslinked, TT-p(Fent-co-TLR7) will localize to B cells, minimizing toxic systemic immune activation. Described herein is a platform that can be easily modified to treat addiction to other drugs and improve the treatment of bacterial infections, HIV, and flu.

The present disclosure is based on the seminal discovery of a polymeric conjugate that induces durable responses to haptens by crosslinking B cell receptors (BCRs) to promote hapten-specific B cell activation. In particular, it was surprisingly discovered herein that polymers achieve greater multivalent hapten display to B cells than proteins and can therefore more efficiently crosslink hapten-specific BCRs. In general, the induction of high quality, robust, durable antibody responses requires that B cells receive the activation signals necessary for B cell activation, maturation, and differentiation into long-lived antibody secreting cells. These signals include BCR crosslinking, T cell signaling from activated CD4⁺ T cells, and B cell activation via the recognition of pathogen associated patterns.

Leveraging these discoveries, in one embodiment, the present disclosure provides a conjugate with an immunogenic carrier protein coupled to a polymer, wherein the polymer includes a first monomeric unit with a first monomer coupled to a hapten and a second monomeric unit with a second monomer coupled to an immune cell receptor agonist that activates B cells. In some aspects, the polymeric conjugate includes a water-soluble random copolymer composed of monomers decorated with a hapten (e.g., an opioid such as fentanyl), an immune cell receptor agonist such as a B cell-activating adjuvant, or a combination thereof. In particular aspects, the water-soluble copolymer includes fentanyl and a TLR7-agonist, and in such cases can be referred to as p(Fent-co-TLR7).

In many aspects, the polymer includes a B cell-activating adjuvant that activates B cells. In some aspects, the B cell activating adjuvant is a toll-like receptor, a pathogen recognition receptor, or a receptor of another pathway that activate B cells. For example, in some aspects the B cell activating adjuvant is a CpG nucleotide, poly(I:C), resiquimod, imiquimod, CL075, loxorbine, M5049, CU-CPT9a, gardiquimod, pluripotin, or MPLA. The B cell-activating adjuvant can be attached to the polymer via a biodegradable linkage.

In many aspects disclosed herein, the polymeric conjugate is non-toxic.

The water-soluble random copolymer (e.g., p(Fent-co-TLR7)) can be coupled to a carrier protein such as tetanus toxin (TT). The coupling can be through a self-immolative linker that releases the carrier protein in an unmodified form (e.g., as depicted in FIG. 1D). The copolymer-carrier protein complex can elicit a robust, durable response against a hapten by efficiently crosslinking BCRs, activating B cell-TLR7 and maximizing the presentation of TT peptides (FIG. 1E). Furthermore, by targeting BCRs, which internalize when crosslinked, the conjugates of the present disclosure localize to B cells, minimizing toxic systemic immune activation. By exploiting this mechanism, the conjugate of the present disclosure can be easily modified to treat addiction to a variety of drugs as well as a range of bacterial and viral infections.

In some aspects, the conjugates of the present disclosure are used to treat opioid addiction. The aim of such a vaccination regimen is to elicit opioid-specific antibodies (α-opioids) that bind to their target opioids (FIG. 1A), preventing their passage into organs and thus averting overdose. The conjugates of the present disclosure generate durable α-opioid responses which are maintained by long-lived antibody-secreting cells (LLASCs). LLASCs are generated upon B cell receptor crosslinking and B cell recognition of signals from activated CD4⁺ T cells (i.e., T cell help) (FIG. 1B). Durable antibody-mediated immune responses (i.e., humoral immunity) are then maintained by these LLASCs and memory B cells. Once generated, LLASCs continuously produce antibodies and can persist in the body for decades and provide lasting immunosurveillance. Although memory B cells do not produce antibodies, they can persist for a lifetime in the body and, upon reencountering their cognate antigen during subsequent vaccinations, differentiate into LLASCs that produce antibodies with a greater affinity than the parent memory B cell.

The ability of the disclosed conjugates to generate robust B cell responses is overviewed in EXAMPLE 2. In this example, the conjugate OVA-p(Fent) induced a higher magnitude fentanyl-specific antibody response than OVA-mFent after the initial vaccination. In addition, by incorporating TLR7/8 into the conjugate, OVA-p(TLR7/8-b-Fent) was designed to specifically activate TLR7/8 in B cells while avoiding broad, systemic immune activation. Indeed, OVA-p(TLR7/8-b-Fent) elicited a more pronounced and higher quality fentanyl-specific antibody response than a polymeric vaccine lacking TLR7/8 incorporation into the polymer, highlighting the efficacy gained by co-delivery and antigen and adjuvant.

In certain embodiments, the present disclosure provides a conjugate that includes an immunogenic carrier protein coupled to a polymer, wherein the polymer includes: i) a first monomeric unit with a first monomer coupled to a hapten, and ii) a second monomeric unit with a second monomer coupled to an immune cell receptor agonist.

The terms “peptide,” “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the term “polymer” refers to any of a class of natural or synthetic substances composed of very large molecules, called macromolecules, that are multiples of simpler chemical units called monomers. Polymers make up many of the materials in living organisms, including, for example, proteins, cellulose, and nucleic acids.

As used herein, the term “carrier protein” refers to a protein that is a type of cell membrane protein involved in facilitated diffusion and active transport of substances out of or into the cell. Carrier proteins are often highly selective, and the complement of carrier proteins in a particular membrane is uniquely specialized to the requirements of the compartment that the membrane surrounds. They are widely expressed on both plasma membrane as well as on membranes of organelles, in cells throughout the body. The two major superfamilies of carrier proteins are the ATP-binding cassette (ABC) superfamily and the solute carrier (SLC) superfamily.

By “immunogenic carrier protein” is meant that the carries protein is capable of inducing immune responses. Immunogenic carrier proteins suitable for coupling to a polymer are known in the art. For example, five carrier proteins have been used in licensed conjugate vaccines: a genetically modified cross-reacting material (CRM) of diphtheria toxin, tetanus toxoid (TT), meningococcal outer membrane protein complex (OMPC), diphtheria toxoid (DT), and H. influenzae protein D (HiD). In some aspects, the carrier protein comprises a CRM, an OMPC, a DT, a TT, a HiD, a Keyhole Limpet Hemocyanin (KLH), a Bovine serum albumin (BSA), Ovalbumin (OVA), or a combination thereof. As used herein a polymer may not be restricted to monomers of the same chemical composition or molecular weight and structure. In some aspects the polymer comprises of one kind of monomer. In other aspects, the polymer comprises two or more different types of monomers; such polymers are known as copolymers.

As used herein, the term “hapten” refers to a small molecule which, when combined with a larger carrier such as a protein, can elicit the production of antibodies which bind specifically to it (in the free or combined state). As described herein a hapten is a substance that can combine with a specific antibody but lacks antigenicity of its own. Many small molecules of Mᵣ<1000 such as toxins, drugs and hormones are not capable of invoking immune response when injected directly into animals. They are thus not immunogenic by themselves and are called haptens. In some aspects, these small-molecular-weight compounds are linked to a large molecule, such as a protein like bovine serum albumin or key-hole limpet haemocyanin to make them immunogenic before injecting into an animal to get the desired antibody. In some aspects, the reactive carboxylic or amino group in a hapten could be used to conjugate it to the protein molecule. In other aspects, a reactive group such as carboxylic acid may be added to a hapten for this purpose. In some aspects, lysine, tyrosine or histidine residues in the carrier proteins may be used for linking. In some aspects, most of the antibody will be generated against the carrier protein, but a good proportion will also be generated against the hapten molecule.

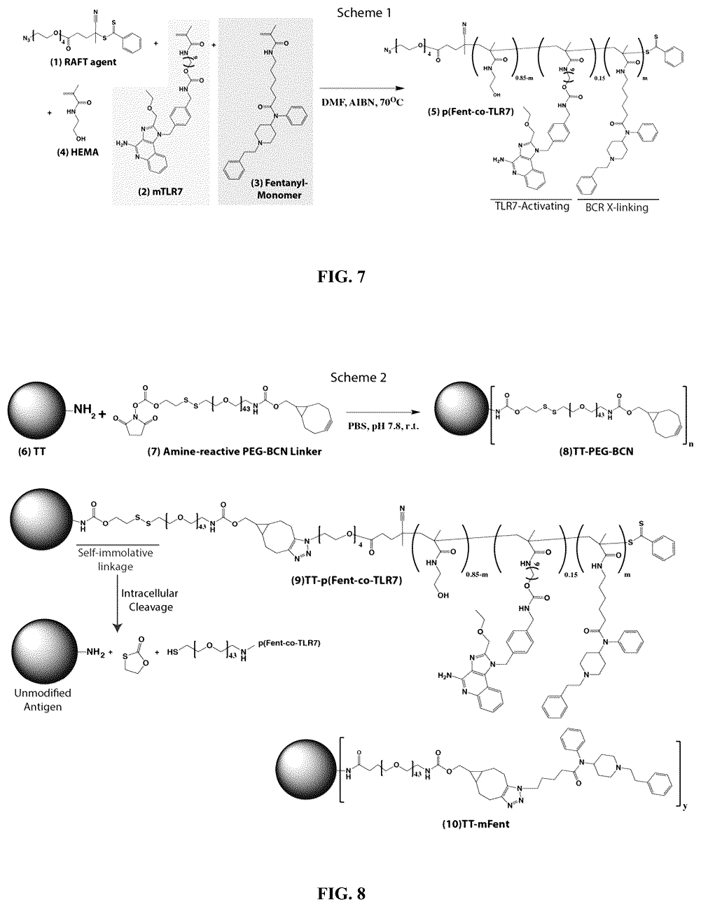

Methods of synthesizing a polymer are known in the art. In some aspects, polymerization or polymer synthesis is a chemical reaction in which monomers are joined together by covalent bonding to form polymer structures. For example, the polymer can be synthesized via the reversible addition fragmentation chain transfer (RAFT) polymerization strategy shown in FIG. 7. In some aspects, the polymer was synthesized via RAFT polymerization. In some aspects, the length of the polymer chain is characterized by the number of repeating units in the chain, which is referred to as degree of polymerization (DP). In certain aspects, the synthesized polymers will have the monomer mole fractions, and DP shown in Table 1.

TABLE 1
Mole fraction of monomers in p(Fent-co-TLR7) polymers

| Polymer | mTLR7 | Fent- | HEMA | DP |
|---|---|---|---|---|
| 1 | 0.15 | 0.1 | 0.75 | 100 |
| 2 | 0.15 | 0.2 | 0.65 | 100 |
| 3 | 0.15 | 0.1 | 0.75 | 200 |
| 4 | 0.15 | 0.1 | 0.65 | 200 |

Methods of generating a polymeric conjugate fentanyl vaccine are known in the art. In some aspects, p(Fent-coTLR7) used in this disclosure was synthesized via RAFT polymerization For example p(Fent-co-TLR7) can be synthesized from an azide-terminated RAFT agent 1, a methacrylamide-modified TLR7-ligand 2 (mTLR7), a methacrylamide-modified fentanyl analogue 3 (fentanyl-monomer), and the biologically inert co-monomer N-(2hydroxyethyl)methacrylamide 4 (HEMA). In some embodiments, compounds 1 and 2 of FIG. 6A-6C are synthesized via procedure disclosed in Wilson, D. S. et al. Antigens reversibly conjugated to a polymeric glyco-adjuvant induce protective humoral and cellular immunity. Nat. Mater. 18, 175-185 (2019), the content of which is hereby incorporated by reference in its entirety. In some embodiments, the fentanyl-monomer is synthesized by modifying a reported procedure for the synthesis of fentanyl analogs. In one aspect, the composition and molecular weight of the p(Fent-co-TLR7) produced for preliminary studies was confirmed via ¹H NMR, C₁₃NMR, and gel permeation chromatography (GPC). In further aspects, each of the polymers will contain between 0.1 and 0.5 mole fraction of mTLR7. In a particular aspect, each of the polymers will contain 0.15 mole fraction of mTLR7. In further aspects, the molar ratio of fentanyl-monomer in p(Fent-co-TLR7) is between 0.01 and 0.2. In a particular aspect, the molar ratio of fentanyl-monomer in p(Fent-co-TLR7) is 0.09.

In particular aspects, the first monomer and the second monomer are each independently an acrylate, an acrylamide, a methacrylate, a methacrylamide, or a vinyl group.

In some aspects, the polymer further includes a third monomeric unit including a third monomer.

In particular aspects, the third monomeric unit does not include a hapten, drug, or biomolecule. In certain aspects, the third monomeric unit has a molecular mass of less than about 250 Da, less than about 225 Da, less than about 200 Da, less than about 175 Da, less than about 150 Da, less than about 125 Da, or less than about 100 Da.

In a particular aspect, the third monomeric unit is an acrylate, an acrylamide, a methacrylate, a methacrylamide, or a vinyl group.

In another aspect, the third monomeric unit is hydroxyethyl methacrylamide.

In certain aspects, the first monomer is identical to the second monomer. In further aspects, the first, second, and third monomer are identical. For examples the polymer can be a homopolymer. In some aspects, the first monomer, the second monomer, and the third monomer are opioid (e.g. fentanyl, a fentanyl analog, buprenorphine, codeine, dextromoramide, dihydrocodeine, enkephalin, heroin, hydrocodone, hydromorphone, meperidine, methadone, morphine, nicomorphine, opium, tramanol, oxycodone, oxymorphone, pentazinine, pentazocine, methamphetamine, a derivative thereof, a precursor thereof, or pharmacologically acceptable salt or solvate thereof. In a particular aspect, the opioid is fentanyl, a derivative or analog thereof, a precursor thereof, or pharmacologically acceptable salt or solvate thereof) based monomers. In non-limiting examples, the polymer includes: (i) a first monomeric unit including an acrylate, (ii) a second monomeric unit including an acrylate, (iii) and a third monomeric unit including an acrylate. In non-limiting examples, the polymer includes: (i) a first monomeric unit including a fentanyl, (ii) a second monomeric unit including a fentanyl, (iii) and a third monomeric unit including a fentanyl.

In one aspect, the first monomeric unit and the second monomeric unit are between about 1% and 50%, between about 1% and 5%, between about 1% and 10%, between about 1% and 15%, between about 1% and 20%, between about 1% and 25%, between about 1% and 30%, between about 1% and 40%, between about 2% and 5%, between about 2% and 10%, between about 2% and 15%, between about 2% and 20%, between about 2% and 25%, between about 2% and 30%, between about 2% and 40%, between about 2% and 50%, between about 5% and 10%, between about 5% and 15%, between about 5% and 20%, between about 5% and 25%, between about 5% and 30%, between about 5% and 40%, between about 5% and 50%, between about 10% and 20%, between about 10% and 30%, between about 10% and 40%, between about 10% and 50%, between about 15% and 25%, between about 15% and 30%, between about 15% and 40%, between about 15% and 50%, between about 20% and 30%, between about 20% and 40%, between about 20% and 50%, between about 25% and 40%, between about 25% and 50%, between about 30% and 40%, or between about 30% and 50% of monomeric units of the polymer.

In another aspect, the first monomeric unit is between about 0.5% and 2.5%, between about 0.5% and 5%, between about 0.5% and 7.5%, between about 0.5% and 10%, between about 0.5% and 15%, between about 0.5% and 20%, between about 0.5% and 25%, between about 1% and 2.5%, between about 1% and 5%, between about 1% and 7.5%, between about 1% and 10%, between about 1% and 15%, between about 1% and 20%, between about 1% and 25%, between about 2.5% and 5%, between about 2.5% and 7.5%, between about 2.5% and 10%, between about 2.5% and 15%, between about 2.5% and 20%, between about 2.5% and 25%, between about 5% and 7.5%, between about 5% and 10%, between about 5% and 15%, between about 5% and 20%, between about 5% and 25%, between about 7.5% and 10%, between about 7.5% and 15%, between about 7.5% and 20%, between about 7.5% and 25%, between about 10% and 15%, between about 10% and 20%, between about 10% and 25%, or between about 15% and 25% of monomeric units of the polymer. In a particular aspect, first monomeric unit is about 9% of the monomeric units of the polymer.

In a further aspect, the second monomeric unit is between about 0.5% and 2.5%, between about 0.5% and 5%, between about 0.5% and 7.5%, between about 0.5% and 10%, between about 0.5% and 15%, between about 0.5% and 20%, between about 0.5% and 25%, between about 1% and 2.5%, between about 1% and 5%, between about 1% and 7.5%, between about 1% and 10%, between about 1% and 15%, between about 1% and 20%, between about 1% and 25%, between about 2.5% and 5%, between about 2.5% and 7.5%, between about 2.5% and 10%, between about 2.5% and 15%, between about 2.5% and 20%, between about 2.5% and 25%, between about 5% and 7.5%, between about 5% and 10%, between about 5% and 15%, between about 5% and 20%, between about 5% and 25%, between about 7.5% and 10%, between about 7.5% and 15%, between about 7.5% and 20%, between about 7.5% and 25%, between about 10% and 15%, between about 10% and 20%, between about 10% and 25%, or between about 15% and 25% of monomeric units of the polymer. In another aspect, the second monomeric unit is about 15% of the monomeric units of the polymer.

In certain aspects, the first, second, and third monomeric units are between about 50% and 80%, between about 50% and 90%, between about 50% and 100%, between about 60% and 80%, between about 60% and 90%, between about 60% and 100%, between about 70% and 80%, between about 70% and 90%, between about 70% and 100%, between about 80% and 90%, between about 80% and 100%, between about 90% and 100%, between about 95% and 100%, about 100%, or 100% of the monomeric units of the polymer.

In one aspect, the polymer is a random copolymer. In another aspect, the first monomer and the second monomer are non-peptidic.

In some aspects, the polymer has a molecular weight of between about 0.1 to 200 kDa, between about 0.1 to 0.5 kDa, between about 0.1 to 1 kDa, between about 0.5 to 5 kDa, between about 0.5 to 10 kDa, between about 1 and 5 kDa, between about 1 and 10 kDa, between about 1 and 20 kDa, between about 1 and 30 kDa, between about 1 and 40 kDa, between about 1 to 50 kDa, between about 2 and 5 kDa, between about 2 and 10 kDa, between about 2 and 20 kDa, between about 2 and 30 kDa, between about 2 and 40 kDa, between about 2 and 50 kDa, between about 5 and 10 kDa, between about 5 and 20 kDa, between about 5 and 30 kDa, between about 5 and 40 kDa, between about 5 and 50 kDa, between about 5 and 100 kDa, between about 10 and 20 kDa, between about 10 and 30 kDa, between about 10 and 40 kDa, between about 10 and 50 kDa, between about 10 and 100 kDa, between about 10 and 200 kDa, between about 20 and 30 kDa, between about 20 and 40 kDa, between about 20 and 50 kDa, between about 20 and 100 kDa, between about 20 and 150 kDa, between about 20 and 200 kDa, between about 30 and 40 kDa, between about 30 and 50 kDa, between about 30 and 100 kDa, between about 30 and 150 kDa, between about 30 and 200 kDa, between about 50 and 100 kDa, between about 50 and 150 kDa, between about 50 and 200 kDa, between about 100 and 150 kDa, or between about 100 and 200 kDa. In a particular aspect, the polymer has a molecular weight of about 24 kDa. The Molecular weight of the polymer can be determined using methods known in the art. For example, the molecular weight of the polymer conjugate can be measured using electrophoresis, MALSI, and GPC.

In certain aspects, the polymer has between about 10 and 5000 monomeric units.

For example, the polymer has about 10, 20, 30, 40, 50, 75, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, or more monomeric units.

In some aspects, the hapten is an opioid. As used herein, the term “opioid” refers to a class of drugs that derive from, or mimic, natural substances found in the opium poppy plant. Opioids work in the brain to produce a variety of effects, including pain relief. Opioid drugs include prescription pain medicine and illegal drugs. An opioid can be a natural opioid, semi-synthetic opioid, or synthetic opioid.

In certain aspects, the opioid is fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, or pharmacologically acceptable salt or solvate thereof. In a particular aspect, the opioid is fentanyl, a derivative or analog thereof, a precursor thereof, or pharmacologically acceptable salt or solvate thereof. In some aspects, the fentanyl analog is acetyl fentanyl, parafluorofentanyl, furanyl fentanyl, or carfentanil. In a specific aspect, the opioid is fentanyl. In some aspects, the hapten is irreversibly coupled to the first monomer.

In another aspect, the hapten is a chemical warfare agent. As used herein the term “chemical warfare agent” refers to extremely toxic synthetic chemicals that can be dispersed as a gas, liquid or aerosol or as agents adsorbed to particles to become a powder. In some aspects, chemical warfare agents have either lethal or incapacitating effects on humans. Non limiting examples of chemical warfare agents include Agent orange, Carbonyl chloride (phosgene) (CG), Diphosgene (DP), Chloropicrin (PS), Tabun (GA), Sarin (GB), Soman (GD), Cyclosarin (GF), O-Ethyl S-(2-diisopropylaminoethyl) methylphosphonothiolate (VX), Novichok (A-230), Novichok (A-232), Novichok (A-234), Hydrogen cyanide (AC), Cyanogen chloride (CK), Sulfur mustard (HD), Nitrogen mustard (HN-1), Nitrogen mustard (HN-2), Nitrogen mustard (HN-3), Lewisite (L), 3-Quinuclidinyl benzilate (BZ)

In some aspects, the chemical warfare agent is agent orange, 3-quinuclinidyl benzilate, VX gas, VR gas, HN1, HN2, or Tabun.

In a further aspect, the hapten is nicotine, cocaine, or MDMA (3,4-Methylenedioxymethamphetamine, commonly known as ecstasy).

In a further aspect, the hapten is a peptide or sugar derived from an infectious agent.

As used herein, the term “infectious agent” is meant to include bacterial agent, viral agent, and parasite agent. The phrase “peptide or sugar derived from” is meant to include any protein, peptide, antigen, or epitope, as well as sugar that can be isolated, purified, or extracted from any of the bacterial, viral, and parasite agents.

The second monomer of the second monomeric unit of the conjugates described herein is coupled to an immune cell receptor agonist.

In certain aspects, the immune cell receptor agonist is an agonist of an intracellular receptor. As used herein, the term “immune cell receptors” refers to receptors in the immune system. Non limiting examples of immune cell receptors include pattern recognition receptors (PRRs), Toll-like receptors (TLRs), killer activated and killer inhibitor receptors (KARs and KIRs), complement receptors, Fc receptors, B cell receptors and T cell receptors. In mammals, Toll-like receptors play a key role in enabling the initiation of adaptive immunity.

In a particular aspect, the immune cell receptor agonist is a toll-like receptor (TLR) agonist. TLRs are expressed on innate immune cells, like macrophages and dendritic cells. They are located on the cell surface or in intracellular compartments. TLRs recognize general microbial patterns, and they are essential for innate immune-cell activation and inflammatory responses. Toll-like receptors (TLRs) recognize pathogen-associated molecular patterns (PAMPs) to activate immunity and inflammatory cascades. Potent TLR ligands or TLR agonists can be used to activate TLR signaling cascades.

In some embodiments, the TLR agonist is one known in the art and/or described herein. The TLR agonists may include an agonist to TLR1, TLR2, TLR3, TLR4; TLR5, TLR6, TLR7, TLR8, TLR9, TLR10 or TLR 11. Non limiting example of TLRs agonist include single stranded RNAs such as ssRNA with 6UUAU repeats, RNA homopolymer (ssPolyU naked), HIV-1 LTR-derived ssRNA (ssRNA40), or ssRNA with 2 nucleotide repeats (ssRNA-DR), TLR7 agonist such as imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, S-34240, SBT-6050, SBT-6290, ZM-TLR8, VX-001, MBS-8, APR-002, Imiquimod VacciGrade™, Gardiquimod VacciGrade™, or Gardiquimod™; adenine analog CL264; base analog CL307; guanosine analog loxoribine; TLR7/8 agonists such as thiazoquinoline compound CL075; imidazoquinoline compound CL097, R848, or R848 VacciGrade™). In certain embodiments, the TLR agonist is a specific agonist listed above. In further embodiments, the TLR agonist is one that agonizes either one TLR or two TLRs specifically. In a further aspect, the immune cell receptor agonist is a toll-like receptor 7 (TLR7) agonist, a toll-like receptor 8 agonist (TLR8), or an agonist of TLR7 and TLR8. In another aspect, the immune cell receptor agonist is a TLR7 agonist.

In one aspect, the TLR7 agonist is imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, 5-34240, SBT-6050, SBT-6290, ZM-TLR8, VX-001, MBS-8, APR-002, or a combination thereof. In another aspect, the TLR7 agonist is an imidazoquinoline. In a further aspect, the TLR7 agonist is

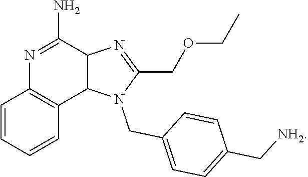 [SMILES: CCOCC1=NC2C(N)=Nc3ccccc3C2N1Cc1ccc(CN)cc1]

Also included herein are conjugates of the invention, which do not include a TLR agonist. Without wishing to be bound to any theory, it is believed that depending on the administered dose, the conjugates described herein could be as potent in the absence of a TLR agonist, pending that the administered dose is increased.

In some aspects, the immune cell receptor agonist is coupled to the second monomer by a cleavable linker. As used herein the term “linker,” which can be cleavable or non-cleavable, refers to a structure that connect two or more molecules together to form a conjugate. As used herein, the term “cleavable linker” refers to a linker that has a chemical trigger in its structure that can be efficiently cleaved to release the two or more molecules. In some aspects, the cleavable linker is an enzyme-cleavable linker or acid-cleavable linker. In some aspects, the cleavable linker is a protease-sensitive peptide linker, an acid sensitive hydrazone linker, or a glutathione-sensitive disulfide linker. Non limiting examples of cleavable linkers include DBCO-NHS ester, Mc-Val-Cit-PABC-PNP, DBCO-PEG4-amine, DSG Crosslinker, MC-Val-Cit-PAB, DBCO-acid, DBCO-amine, DSP Crosslinker, MC-Gly-Gly-Phe, Val-cit-PAB-OH, Fmoc-NH-PEG4-CH2CH2COOH, SPDP, MAC glucuronide linker-2, Fmoc-3VVD-OH, Fmoc-Gly-Gly-Phe-OH, Fmoc-Val-Cit-PAB-PNP, DBCO-PEG5-NHS ester, Fmoc-Val-Cit-PAB, 6-Azido-hexylamine, Fmoc-8-amino-3,6-dioxaoctanoic acid, sulfo-SPDB, Boc-NH-C6-Br, N-Butanoyl-L-homoserine lactone, Boc-Dap-NE, N-Boc-PEG4-bromide, SPDB, TCO-PEG4-NHS ester, Boc-NH-PEG4-CH2CH2NH2, β-D-glucuronide-pNP-carbonate, Fmoc-PEA, Fmoc-Val-Ala-PAB-PNP, N-Boc-PEG1-bromide, N-Boc-PEG2-bromide, Gly-Gly-Gly-PEG4-azide, MethylCBI-azaindole-benzamide-MOM-Boc-ethylenediamine-D, Azido-PEG8-NHS ester, Bis-PEG5-NHS ester, Biotin-PEG1-NH2, Fmoc-NH-PEG6-CH2CH2COOH, CL2A, Azido-PEG3-Val-Cit-PAB-PNP, NHS-SS-Ph, SPDMV, DBCO-CONH-S-S-NHS ester, Boc-Val-Cit-PAB-PNP, Boc-Gly-Gly-Phe-Gly-OH, Fmoc-Asp-NH2, Fmoc-Val-Ala-PAB-OH, Gly-Gly-Gly-PEG4-DBCO, N-Boc-PEG5-bromide, MC-Gly-Gly-Phe-Gly, Fmoc-Glu-(Boc)-Val-Cit-PAB-PNP, Mal-PEG1-NHS ester, Fmoc-NH-PEG3-CH2CH2COOH, Boc-Val-Cit-OH, NH2-PEG4-CH2CH2COOH, PDEC-NB, Fmoc-NH-PEG8-CH2CH2COOH, Fmoc-Gly-Gly-OH, DBCO-Sulfo-NHS ester sodium, SPP, Biotin-PEG3-aldehyde, Azido-PEG6-NHS ester, m-PEG8-Amine, Amino-PEG3-C2-acid, O-Estradiol-6-CMO-PEG3-biotin, Bis-PEG9-NHS ester, MAC glucuronide linker-1, m-PEG2-Amine, S-(1-Hydroxy-2-methylpropan-2-yl) methanesulfonothioate, Cyclooctyne-O-NHS ester, Tetrazine-biotin, Azido-PEG5-CH2CO2H, Amino-PEG2-C2-acid, DBCO-PEG4-DBCO, BCN-SS-NHS, PPC-NB, Mc-Gly-Gly-Phe-Gly-PAB-OH, Bis-PEG13-NHS ester, Azide-C2-SS-C2-biotin, Azido-C6-OH, Br-PEG4-C2-Boc, MC-Val-Ala-PAB-PNP, Propargyl-PEG3-NHS ester, Mal-amido-PEG2-Val-Cit-PAB-PNP, N-Boc-PEG6-alcohol, Aminoethyl-SS-propionic acid, DTSSP Crosslinker, DSS Crosslinker, DBCO-NHS ester, Mc-Val-Cit-PABC-PNP, Sulfo-SMCC sodium, DBCO-PEG4-amine, DSG Crosslinker, MC-Val-Cit-PAB, DBCO-acid, DBCO-amine, 6-Maleimidohexanoic acid N-hydroxysuccinimide ester, DSP Crosslinker, MC-Gly-Gly-Phe, Maleimide-DOTA, N3-PEG4-C2-NHS ester, Val-cit-PAB-OH, Fmoc-NH-PEG4-CH2CH2COOH, SPDP, N-Hydroxysulfosuccinimide sodium, MAC glucuronide linker-2, Fmoc-3VVD-OH, Fmoc-Gly-Gly-Phe-OH, BS3 Crosslinker, Azido-PEG2-C2-amine, Fmoc-Val-Cit-PAB-PNP, BS3 Crosslinker disodium, DBCO-PEG5-NHS ester, Fmoc-Val-Cit-PAB, 6-Azido-hexylamine, Fmoc-8-amino-3,6-dioxaoctanoic acid, sulfo-SPDB, Boc-NH-C6-Br, N-Butanoyl-L-homoserine lactone, Boc-Dap-NE, Vipivotide tetraxetan Ligand-Linker Conjugate, N-Boc-PEG4-bromide, SPDB, TCO-PEG4-NHS ester, Mal-PEG4-NHS ester, Boc-NH-PEG4-CH2CH2NH2, Mal-PEG2-NHS ester, N3-PEG3-CH2CH2COOH, β-D-glucuronide-pNP-carbonate, N3-PEG2-C2-NHS ester, NH2-PEG2-C2-Boc, Fmoc-PEA, N3-PEG3-C2-NHS ester, BMPS, Boc-C16-COOH, Bromo-PEG2-C2-azide, Methyl azetidine-3-carboxylate hydrochloride, DBCO-Maleimide, Fmoc-Val-Ala-PAB-PNP, Azido-PEG6-amine, Azido-PEG7-amine, Fluorescein-DBCO, N-Boc-PEG1-bromide, N-Boc-PEG2-bromide, Gly-Gly-Gly-PEG4-azide, MethylCBI-azaindole-benzamide-MOM-Boc-ethylenediamine-D, Azido-PEG8-NHS ester, Bis-PEG5-NHS ester, Boc-Hyp-OH, Mal-PEG3-NHS ester, Amino-PEG4-alcohol, Fmoc-Lys-OH hydrochloride, Biotin-PEG1-NH2, Fmoc-NH-PEG6-CH2CH2COOH, CL2A, Azido-PEG3-Val-Cit-PAB-PNP, NHS-SS-Ph, SPDMV, DBCO-CONH-S-S-NHS ester, Boc-Val-Cit-PAB-PNP, Boc-Gly-Gly-Phe-Gly-OH, BCN-PEG3-Biotin, Mal-amido-PEG8-C2-acid, Mal-PEG6-NHS ester, NH2-PEG8-acid, Propargyl-PEG1-NHS ester, 1-Boc-azetidine-3-carboxylic acid, Fmoc-Asp-NH2, Succinic anhydride, DBCO-PEG3-oxyamine, Mal-C2-NHS ester, Fmoc-Val-Ala-PAB-OH, Gly-Gly-Gly-PEG4-DBCO, N-Boc-PEG5-bromide, MC-Gly-Gly-Phe-Gly, Fmoc-Glu-(Boc)-Val-Cit-PAB-PNP, N3-C3-NHS ester, DBCO-Val-Cit-PABC-PNP, AMAS, Bis-PEG2-NHS ester, H-Glu-OtBu, Mal-PEG1-NHS ester, Fmoc-NH-PEG3-CH2CH2COOH, Boc-Val-Cit-OH, NH2-PEG4-CH2CH2COOH, PDEC-NB, Azetidin-3-ol hydrochloride, Fmoc-NH-PEG8-CH2CH2COOH, N3-C5-NHS ester, Fmoc-NH-PEG2-CH2CH2COOH, Aminooxy-PEG2-azide, Fmoc-Gly-Gly-OH, m-PEG5-CH2COOH, DBCO-Sulfo-NHS ester sodium, SPP, Biotin-PEG3-aldehyde, Azido-PEG6-NHS ester, m-PEG8-Amine, Propargyl-PEG2-acid, Amino-PEG3-C2-acid, β-Estradiol-6-CMO-PEG3-biotin, Bis-PEG9-NHS ester, m-PEG12-OH, 20-(tert-Butoxy)-20-oxoicosanoic acid, MAC glucuronide linker-1, m-PEG2-Amine, S-(1-Hydroxy-2-methylpropan-2-yl), methanesulfonothioate, N3-C2-NHS ester, 1-N-Boc-3-hydroxyazetidine, Cyclooctyne-O-NHS ester, Biotin-PEG2-acid, Tetrazine-biotin, NH2-PEG6-Boc, Azido-PEG5-CH2CO2H, Amino-PEG2-C2-acid, DBCO-PEG4-DBCO, BCN-SS-NHS, PPC-NB, Mc-Gly-Gly-Phe-Gly-PAB-OH, Bis-PEG13-NHS ester, Azide-C2-SS-C2-biotin, Azido-C6-OH, Br-PEG4-C2-Boc, DBCO-PEG4-Maleimide, Propargyl-PEG2-amine, Mal-PEG1-acid, m-PEG7-CH2CH2COOH, Bis-PEG3-NHS ester, MC-Val-Ala-PAB-PNP, Propargyl-PEG3-NHS ester, Mal-amido-PEG2-Val-Cit-PAB-PNP, Propargyl-C1-NHS ester, N-Boc-PEG6-alcohol, Mal-amido-PEG2-NHS ester, Aminoethyl-SS-propionic acid, DTSSP Crosslinker, Azido-PEG5-alcohol, N-Boc-cis-4-hydroxy-L-proline, Propargyl-PEG4-NHS ester, Hydroxy-PEG3-(CH2)2-Boc, NH2-PEG6-CH2CH2COOH, Bis-PEG7-NHS ester, NHS-SS-biotin, N-Boc-diethanolamine, endo-BCN-PEG4-acid, Bis-PEG4-NHS ester, Azido-PEG4-Val-Cit-PAB-OH, Amino-PEG6-alcohol, Propargyl-PEG5-NHS ester, Mal-PEG2-acid, Mc-Val-Ala-PAB, Mal-PEG2-VCP-NB, 4-Methyl-4-(methyldisulfanyl)pentanoic acid, m-PEG12-amine, PC Mal-NHS carbonate ester, Fmoc-NH-PEG1-CH2COOH, Azido-PEG9-amine, Mc-Val-Cit-PAB-C1, DBCO-C6-acid, Docosanedioic acid, NHS-PEG2-SS-PEG2-NHS, Mal-L-PA-NH-Boc.

In certain aspects, the cleavable linker includes a carbamate. In one application, linkers include compounds for molecular conjugation reactions to provide structural stability or assistance in protein-protein, protein-peptide, protein-polymer, polymer-small molecule, peptide/protein-small molecule interactions, immobilization for assays or purification, as well as various peptide-nucleic acid and nucleic acid-nucleic acid conjugations, among many others. Typically, linkers contain functional groups, such as primary amines, sulfhydryls, acids, alcohols, azides, alkynes and halides. Without intending to be limited to theory, the present disclosure also encompasses all cleavable linkers used in chemical biology classified according to their cleavage conditions by enzymes, nucleophilic/basic reagents, reducing agents, photo-irradiation, electrophilic/acidic reagents, organometallic and metal reagents, or oxidizing reagents. In some embodiments, the self-immolative linkage is known to be relatively stable in serum, but cleaves upon endocytosis as described in Yang, J., Chen, H., Vlahov, I. R., Cheng, J. X. & Low, P. S. Evaluation of disulfide reduction during receptor-mediated endocytosis by using FRET imaging. Proc. Natl. Acad. Sci. U.S.A 103, 13872-13877 (2006), the content of which is hereby incorporated by reference in its entirety.

In some aspects, the immune cell receptor is coupled to the second monomer by a bifunctional linker. As used herein, the term “bifunctional linker” refers to a molecule with two or more functional groups that are used to cross-link two or more molecules together. Types of bifunctional crosslinkers include homobifunctional crosslinkers, heterobifunctional crosslinkers, and photoreactive crosslinkers. Reasons to use a bifunctional crosslinker include the ability to control the size of conjugates, increased conjugate stability and improved detection and ability to analyze conjugates once they are created. Methods of synthesizing a bifunctional linker are known in the art. Exemplary compounds used as bifunctional linkers in the preparation of the polymeric conjugated vaccines of the current invention including any of the above mentioned functional groups can include alkyne-PEG5-acid, N-alloc-1,4-butandiamine hydrochloride, N-alloc-1,6-hexanediamine hydrochloride, allyl(4-methoxyphenyl)dimethylsilane, 6-(allyloxycarbonylamino)-1-hexanol, 3-(allyloxycarbonylamino)-1-propanol, 4-aminobutyraldehyde diethyl acetal, (E)-N-(2-aminoethyl)-4-{2-[4-(3-azidopropoxy)phenyl]diazenyl}benzamide hydrochloride, N-(2-aminoethyl)maleimide trifluoroacetate salt, amino-PEG4-alkyne, benzyl N-(3-hydroxypropyl)carbamate, 4-(Boc-amino)-1-butanol, 4-(Boc-amino)butyl bromide, 2-(Boc-amino)ethanethiol, 2-[2-(Boc-amino)ethoxy]ethoxyacetic acid, (dicyclohexylammonium) salt, 2-(Boc-amino)ethyl bromide, 6-(Boc-amino)-1-hexanol, 21-(Boc-amino)-4,7, 10, 13,16, 19-hexaoxaheneicosanoic acid, 6-(Boc-amino)hexyl bromide, 5-(Boc-amino)-1-pentanol, 3-(Boc-amino)-1-propanol, 3-(Boc-amino)propyl bromide, 15-(Boc-amino)-4,7, 10,13-tetraoxapentadecanoic acid, N-Boc-1,4-butanediamine, N-Boc-cadaverine, N-Boc-ethanolamine, N-Boc-ethylenediamine, N-Boc- 2,2′-(ethylenedioxy)diethylamine, N-Boc-1,6-hexanediamine, N-Boc-1,6-hexanediamine hydrochloride, N-Boc-4-isothiocyanatoaniline, N-Boc-4-isothiocyanatobutylamine, N-Boc-2-isothiocyanatoethylamine, N-Boc-3-isothiocyanatopropylamine, N-Boc-N-methylethylenediamine, N-Boc-m-phenylenediamine, N-Boc-p-phenylenediamine, 2-(4-Boc-1-piperazinyl)acetic acid, N-Boc-1,3-propanediamine, N-Boc-1,3-propanediamine, N-Boc-N′-succinyl-4,7, 10-trioxa-1,13-tridecanediamine, N-Boc-4,7,10-trioxa-1, 13-tridecanediamine, N-(4-Bromobutyl)phthalimide, 4-bromobutyric acid, 4-bromobutyryl chloride, 4-bromobutyryl chloride, N-(2-bromoethyl)phthalimide, 6-bromo-1-hexanol, 3-(bromomethyl)benzoic acid N-succinimidylester, 4-(bromomethyl)phenyl isothiocyanate, 8-bromooctanoic acid, 8-bromo-1-octanol, 4-(2-bromopropionyl)phenoxyacetic acid, N-(3-bromopropyl)phthalimide, 4-(tert-Butoxymethyl)benzoic acid, tert-butyl 2-(4-{[4-(3-azidopropoxy)phenyl]azo}benzamido)ethylcarbamate, 2-[2-(tert-butyldimethylsilyloxy)ethoxy]ethanamine, tert-butyl 4-hydroxybutyrate, chloral hydrate, 4-(2-chloropropionyl)phenylacetic acid, 1, 1 1-diamino-3,6,9-trioxaundecane, di-Boc-cystamine, di ethylene glycol monoallyl ether, 3,4-Dihydro-2H-pyran-2-methanol, 4-[(2,4-Dimethoxyphenyl)(Fmoc-amino)methyl]phenoxyacetic acid, 4-(Diphenylhydroxymethyl)benzoic acid, 4-(Fmoc-amino)-1-butanol, 2-(Fmoc-amino)ethanol, 2-[2-(Fmoc-amino)ethoxy]ethylamine hydrochloride, 2-(Fmoc-amino)ethyl bromide, 6-(Fmoc-amino)-1-hexanol, 5-(Fmoc-amino)-1-pentanol, 3-(Fmoc-amino)-1-propanol, 3-(Fmoc-amino)propyl bromide, N-Fmoc-2-bromoethylamine, N-Fmoc-1,4-butanediamine hydrobromide, N-Fmoc-cadaverine hydrobromide, N-Fmoc-ethylenediamine hydrobromide, N-Fmoc-1,6-hexanediamine hydrobromide, N-Fmoc-1,3-propanediamine hydrobromide, N-Fmoc-N″-succinyl-4,7, 10-trioxa-1,13-tridecanediamine, (3-Formyl-1-indolyl)acetic acid 6-Guanidinohexanoic acid 4-Hydroxybenzyl alcohol N-(4-hydroxybutyl)trifluoroacetamide, 4′-hydroxy-2,4-dimethoxybenzophenone, N-(2-hydroxyethyl)maleimide, 4-[4-(1-hydroxy ethyl)-2-methoxy-5-nitrophenoxy]butyric acid, N-(2-hydroxyethyl)trifluoroacetamide, N-(6-hydroxyhexyl)trifluoroacetamide, 4-hydroxy-2-methoxybenzaldehyde, 4-hydroxy-3-methoxybenzyl alcohol, 4-(hydroxymethyl)benzoic acid, 4-hydroxymethyl-3-methoxyphenoxyacetic acid, 4-(4-hydroxymethyl-3-methoxyphenoxy)butyric acid, 4-(hydroxymethyl)phenoxyacetic acid, 3-(4-hydroxymethylphenoxy)propionic acid, N-(5-hydroxypentyl)trifluoroacetamide, 4-(4′-hydroxyphenylazo)benzoic acid, N-(3-hydroxypropyl)trifluoroacetamide, 2-maleimidoethyl mesylate, 4-mercapto-1-butanol, 6-mercapto-1-hexanol, phenacyl 4-(bromomethyl)phenylacetate, phenacyl 4-(bromomethyl)phenylacetate, 4-sulfamoylbenzoic acid, 4-sulfamoylbutyric acid, N-trityl-1,2-ethanediamine hydrobromide, 4-(Z-amino)-1-butanol, 6-(Z-amino)-1-hexanol, 5-(Z-amino)-1-pentanol, N-Z-1,4-butanediamine hydrochloride, N-Z-ethanolamine, N-Z-ethylenediamine hydrochloride, N-Z-ethylenediamine hydrochloride, N-Z-1,6-hexanediamine hydrochloride, N-Z-1,5-pentanediamine hydrochloride, and N-Z-1,3-propanediamine hydrochloride. Non-limiting examples of trifunctional linkers used to link three separate molecules together include N1,N4-bis-Boc-spermidine, N1,N5-bis-Boc-spermidine, N-Boc-diethanolamine, N1-Boc-2,2′-iminodiethylamine, N-Boc-iminodipropionic acid, N1-Boc-3,3′-iminodipropylamine, N,N″-Di-Z-diethylenetriamine. In specific aspects, the bifunctional linker contains a radical conjugation functional group, such as found in 2-(2-(2-(2-azidoethoxy)ethoxy)ethoxy)ethyl 4-cyano-4-(phenylcarbonothioylthio)pentanoate that can be first conjugated with monomers in a polymerization reaction (i.e., reversible addition-fragmentation chain transfer (RAFT) polymerization) to afford an azide functionalized agent. The azide agent can then be used in subsequent conjugation reactions to prepare polymer conjugate vaccines or polymer conjugate vaccine precursors. In one non-limiting example, the bifunctional linker is the poly(ethylene glycol) linker 7 functionalized with an amine-reactive NHS-carbonate and bicyclononyne (BCN)—a copper-free click ligand that spontaneously reacts with azides and was synthesized as described in Wilson, D. S. et al. Antigens reversibly conjugated to a polymeric glyco-adjuvant induce protective humoral and cellular immunity. Nat. Mater. 18, 175-185 (2019), the content of which is hereby incorporated by reference in its entirety.

In some aspects, the immunogenic carrier protein is a pathogenic protein. As used herein the term “pathogenic protein” refers to a protein produced by a pathogen. Pathogens are organisms that can cause disease. The different types of pathogens and the severity of the diseases that they cause are very diverse. In some aspects, the pathogenic protein is a bacterial protein, a viral protein, a fungal protein, protein from protists, or protein from parasitic warms.

In a particular aspect, the immunogenic carrier protein is a bacterial protein. Non limiting examples of bacterial proteins that can be used as immunogenic carrier proteins included tetanus toxins, diphtheria toxins, related proteins such as CRM197, a genetically toxoided variant of diphtheria toxin, or recombinant P. aeruginosa exoprotein.

In one aspect, the immunogenic carrier protein is a tetanus protein. In another aspect, the immunogenic carrier protein is a tetanus toxin or other immunogenic protein. As used herein the term “tetanus toxin” refers to a toxin produced by the clostridial family of bacterium. In some aspects, the tetanus toxin is an inactivated tetanus toxin. Inactivated toxins are made by purifying the bacterial exotoxin. Toxicity of purified exotoxins is then suppressed or inactivated either by heat or with formaldehyde (while maintaining immunogenicity) to form inactivated toxins (toxoids). Formalin, formaldehyde, and sterilized water treatment are ways which detoxify and inactivate toxins.

In some aspects, TT-p(Fent-co-TLR7) conjugates will be made with polymers 1-4 in Table 1.

In one aspect, the immunogenic carrier protein is a diphtheria toxin. Diphtheria is a disease caused by the exotoxin (diphtheria toxin) of the facultative anaerobic bacterium Corynebacterium diphtheriae. In some aspects, the diphtheria toxin is a detoxified diphtheria. Toxicity of purified exotoxins is then suppressed or inactivated either by heat or with formaldehyde (while maintaining immunogenicity) to form inactivated toxins (toxoids). Formalin, formaldehyde, and sterilized water treatment are ways which detoxify and inactivate toxins. In some aspects, the detoxified diphtheria is prepared by formaldehyde treatment of toxin and is standardized for potency according to the specifications of the US Food and Drug Administration. While removing the toxicity, the treatment also changes the epitopes of the molecule with a potential reduction of the immunogenicity and protective efficacy of the detoxified diphtheria. To avoid the issue, a number of mutants that had lost toxicity were generated. These proteins were called cross-reacting material (CRM), since they were immunologically related to diphtheria toxin.

Of the different diphtheria toxin mutants obtained by nitrosoguanidine or by site-directed mutagenesis, CRM197 is the most popular.

In another aspect, the immunogenic carrier protein is a genetically detoxified diphtheria toxin. In a further aspect, the immunogenic carrier protein is a diphtheria toxin. In a particular aspect, the immunogenic carrier protein is CRM197. CRM197 is a genetically detoxified form of diphtheria toxin. A single mutation at position 52, substituting glutamic acid for glycine, causes the ADP-ribosyltransferase activity of the native toxin to be lost. The polymer conjugate can be purified via any methods know in the art or described herein. For example, the polymer conjugate can be purified via chromatography, centrifugation, dialysis, extraction, recrystallization, distillation, or a combination thereof. In one aspect the polymer conjugate can be purified using size exclusion chromatography (SEC).

In some aspects, the immunogenic carrier protein coupled to the polymer by a cleavable linker. In one such aspect, the cleavable linker is a self-immolating linker. As used herein the term “self-immolating linker” refers to a linker that undergoes a spontaneous and irreversible disassembly into its constituent fragments through either an electronic cascade, via an elimination pathway or, alternatively, where self-immolation is achieved through a cyclisation-elimination event. Self-immolating linkers can be cleaved by factors including responding to the difference between the extracellular and intracellular environments (pH, ROS, glutathione) through over-expressed enzymes (cathepsin, plasmin, β-glucuronidase) or biorthogonal activation. The mechanism for self-immolation involves the linker undergoing a 1,4- or 1,6-elimination (via electron cascade) or intramolecular cyclisation to release cytotoxic drug at the targeted site.

In another aspect, cleavage of the cleavable linker releases the immunogenic carrier protein in an unmodified form.

In some aspects, the conjugate is water-soluble. In further aspects, the conjugate is non-toxic. In additional aspects, the conjugate is configured to internalize within a B cell. In particular aspects, the B cell is an opioid-specific B cell. As used herein the term “opioid specific B cell” refers to a B cell that is specific activated in response to opioids. An opioid-specific B cell can recognize opioid molecules and produce antibodies against such molecules. In nonlimiting examples the opioid molecule recognized by the B cell is fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, or pharmacologically acceptable salt or solvate thereof. In a particular aspect, the opioid is fentanyl, a derivative or analog thereof, a precursor thereof, or pharmacologically acceptable salt or solvate thereof.

In a further embodiment, the present disclosure provides a pharmaceutical composition that includes the conjugate of the present disclosure and a pharmaceutically acceptable excipient.

As used herein, “pharmaceutical composition” refers to a formulation comprising an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. In one embodiment, the active ingredient includes a biologically active molecule. As used herein, the phrase “biologically active molecule” refers to a molecule that has a biological effect in a cell. In certain embodiments the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, a nucleic acid such as a DNA or RNA molecule, a ribozyme, hairpin RNA, siRNA (small interfering RNAs) of varying chemistries, miRNA, siRNA-protein conjugate, an siRNA-peptide conjugate, and siRNA-antibody conjugate, an antagomir, a PNA (peptide nucleic acid), an LNA (locked nucleic acids), or a morpholino. In certain illustrative embodiments, the active agent is a polypeptide or peptide.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil, and dimethyl sulfoxide (DMSO).

In another embodiment, the present disclosure provides a method of treating addiction in a subject in need thereof that includes administering the conjugate or the composition of the present disclosure to the subject, thereby treating the addiction in the subject. In some aspects, the addiction is opioid addiction.

The term “subject” as used herein refers to any individual or patient to which the subject methods are performed. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., immune activation). Such amount should be sufficient to elicit opioid-specific antibodies and prevent or treat drug abuse. The effective amount can be determined as described herein.

The terms “administration of” and or “administering” should be understood to mean providing a pharmaceutical composition in a therapeutically effective amount to the subject in need of treatment. In some aspects, administration can be via inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic administration, or any combination thereof.

In a further embodiment, the present disclosure provides a method of preventing drug overdose in a subject in need thereof comprising administering the conjugate or the composition of the present disclosure to the subject, thereby preventing the overdose in the subject. In some embodiments, the overdose is an opioid overdose.

As used herein, the terms “drug overdose,” “overdose” or “OD” refer to the ingestion or application of a drug or other substance in quantities much greater than are recommended. An overdose may result in a toxic state or death. The word “overdose” implies that there is a common safe dosage and usage for the drug; therefore, the term is commonly applied only to drugs, not poisons. Usage of illicit drugs, in large quantities, or after a period of drug abstinence can induce overdose.

By “preventing overdose” it is meant that the conjugate of composition of the present invention can (i) prevent the symptoms of an overdose to occur, when administered prior to the occurrence of an overdose, (ii) treat and/or alleviate the symptoms of the overdose, when administered when the first signs of an overdose are detected, and (iii) prevent the risk associated with an overdose, thereby implying that the conjugate can decrease for example the risk of death from overdose.

In some embodiments, the composition comprises a polymeric opioid conjugate vaccine. polymeric opioid conjugate vaccine induces durable α-opioid responses in the absence of toxicity.

In some embodiments, the present disclosure provides a vaccine composition including a conjugate including an immunogenic carrier protein coupled to a polymer, wherein the polymer includes: a first monomeric unit including a first monomer coupled to a hapten, and a second monomeric unit including a second monomer coupled to an immune cell receptor agonist.

According to the present disclosure, the term “vaccine” relates to a pharmaceutical preparation (pharmaceutical composition) or product that upon administration induces an immune response, in particular a cellular immune response, which recognizes and attacks a pathogen, a diseased cell such as a cancer cell, or a hapten. A vaccine may be used for the prevention or treatment of a disease or conditions. For example, there are vaccines to protect against diseases caused by: viruses or bacteria (e.g., tetanus, diphtheria, and pertussis).

The term “immune response” refers to an integrated bodily response to an antigen and preferably refers to a cellular immune response or a cellular as well as a humoral immune response. The immune response may be protective/preventive/prophylactic and/or therapeutic.

In some embodiments, the present disclosure provides a method of vaccinating a subject in need thereof against substance abuse including administering to the subject a vaccine composition including an immunogenic carrier protein coupled to a polymer, wherein the polymer includes: a first monomeric unit including a first monomer coupled to a hapten, a second monomeric unit including a second monomer coupled to an immune cell receptor agonist, and a third monomeric unit comprising a third monomer, thereby vaccinating the subject against substance abuse.

In some aspects, the immunogenic carrier protein coupled to a polymer is OVA, the hapten is fentanyl, and the immune cell receptor agonist is a TLR7 agonist, a TLR8 agonist, or a TLR7/TLR8 agonist.

In some aspects, the immunogenic carrier protein coupled to a polymer is CRM197, the hapten is fentanyl, and the immune cell receptor agonist is a TLR7 agonist, a TLR8 agonist, or a TLR7/TLR8 agonist.

In some aspects, the immunogenic carrier protein is Keyhole Limpet Hemocyanin (KLH), the hapten is fentanyl, and the immune cell receptor agonist is TLR7 agonist, a TLR8 agonist, or a TLR7/TLR8 agonist.

Types of vaccines include but are not limited to live-attenuated vaccines, inactivated vaccines, subunit, recombinant, polysaccharide, and conjugate vaccines (use only specific pieces of the pathogen, such as its protein, sugar, or casing), toxoid vaccines that (e.g. use a toxin made by the pathogen), mRNA vaccines, viral vector vaccines.

Vaccines are injections (shots), liquids, pills, or nasal sprays that are administered to induce an immune response in a subject. In the context of the present invention, the vaccine aims at inducing a protective immune response against a drug susceptible of causing an overdose. Therefore, the vaccines described herein are to prevent substance abuse.

As used herein the term “substance abuse” refers to a pattern of drug use that leads to significant problems such as failure to attend work or school, driving a vehicle while etc. Substance abuse is the use of illegal drugs or the use of prescription or over-the-counter drugs or alcohol for purposes other than those for which they are meant to be used, or in excessive amounts. Substance abuse may lead to social, physical, emotional, and job-related problems. In some aspects, the substance abuse is drug abuse. In further aspects, the substance abuse is opioid abuse. In a particular aspect, the substance abuse is fentanyl abuse.

“Inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response.” Preferably, after inducing an immune response in a subject, said subject is protected from developing a condition such as a drug overdose or the condition is lessened by inducing an immune response, and limiting and/or reducing the amount of drug circulating in the subject's bloodstream. For example, an immune response against an illicit drug may be induced in a patient susceptible of having on overdose such as subjects consuming illicit drugs.

In some aspects, the hapten is an opioid selected from the group consisting of fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, and pharmacologically acceptable salt or solvate thereof.

In some aspects, the immune cell receptor agonist is a toll-like receptor (TLR) agonist selected from the group consisting of imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, S-34240, SBT-6050, SBT-6290, ZM-TLR8, VX-001, MBS-8, APR-002, imidazoquinoline,

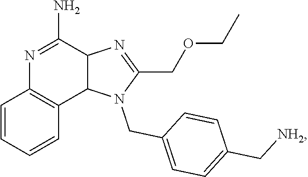 [SMILES: CCOCC1=NC2C(N)=Nc3ccccc3C2N1Cc1ccc(CN)cc1]

and fragment thereof.

In some aspects, the immunogenic carrier protein is selected from the group consisting of tetanus toxins, diphtheria toxins, related proteins such as CRM197, genetically toxoided variant of diphtheria toxin, meningococcal outer membrane protein complex (OMPC), H. influenzae protein D (HiD), Keyhole Limpet Hemocyanin (KLH), Bovine serum albumin (BSA), Ovalbumin (OVA), and recombinant P. aeruginosa exoprotein.

In some embodiments, the present disclosure provides a vaccine composition comprising a conjugate comprising Ovalbumin coupled to a polymer, wherein the polymer includes: a first monomeric unit comprising a first monomer coupled to fentanyl, a second monomeric unit including a second monomer coupled to a TLR7 agonist, a TLR8 agonist, or a TLR7/TLR8 agonist, and a third monomeric unit including a third monomer.

In some embodiments, the present disclosure provides a vaccine composition comprising a conjugate comprising CRM197 coupled to a polymer, wherein the polymer includes: a first monomeric unit including a first monomer coupled to fentanyl, a second monomeric unit comprising a second monomer coupled to a TLR7 agonist, a TLR8 agonist, or a TLR7/TLR8 agonist, and a third monomeric unit including a third monomer.

In some embodiments, the present disclosure provides a method of preventing drug overdose in a subject in need thereof including administering to the subject a conjugate including an immunogenic carrier protein coupled to a polymer, wherein the polymer includes: a first monomeric unit including a first monomer coupled to a hapten, and a second monomeric unit including a second monomer coupled to an immune cell receptor agonist, thereby preventing drug overdose in the subject.

In some aspects, the immunogenic carrier protein coupled to a polymer is OVA, the hapten is fentanyl, and the immune cell receptor agonist is a TLR7 agonist, a TLR8 agonist, or a TLR7/TLR8 agonist.

In some aspects, the immunogenic carrier protein coupled to a polymer is CRM197, the hapten is fentanyl, and the immune cell receptor agonist is a TLR7 agonist, a TLR8 agonist, or a TLR7/TLR8 agonist.

some aspects, the immunogenic carrier protein coupled to a polymer is Keyhole Limpet Hemocyanin (KLH), the hapten is fentanyl, and the immune cell receptor agonist a TLR7 agonist, a TLR8 agonist, or a TLR7/TLR8 agonist.

Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the conjugate, or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the brain, and more specifically neuronal cells. The liposomes will be targeted to and taken up selectively by the desired tissue. Also included in a targeted drug delivery system is nanoparticle specific brain delivery of conjugate or composition, alone or in combination with similar compounds. A summary of various delivery methods and techniques of polymeric conjugate vaccine administration is provided in J Wallis, D P Shenton, R C Carlisle, Novel approaches for the design, delivery and administration of vaccine technologies, Clinical and Experimental Immunology, Volume 196, Issue 2, 189-204 (2019) the content of which is hereby incorporated by reference in its entirety.

One of ordinary skill in the art will appreciate that a method of administering pharmaceutically effective amounts of the pharmaceutical compositions of the invention to a subject in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosages can also be administered in a patient-specific manner to provide a predetermined concentration of the agents in the blood, as determined by techniques accepted and routine in the art.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

In some aspects administration can be in combination with one or more additional therapeutic agents. The phrases “combination therapy,” “combined with” and the like refer to the use of more than one medication or treatment simultaneously to increase the response. The composition of the present invention might for example be used in combination with other drugs or treatment in use to treat or prevent drug use. Specifically, the administration of a polymeric conjugate vaccine to a subject can be in combination with a drug used to treat drug use (e.g., an opioid agonist). Such therapies can be administered prior to, simultaneously with, or following administration of the composition of the present invention.

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

In certain embodiments, the compositions disclosed herein are formulated with additional agents that promote entry into the desired cell or tissue. Such additional agents include micelles, liposomes, and dendrimers.

In addition to the various embodiments described in the specification above, the following additional embodiments are contemplated herein.

1. A conjugate comprising an immunogenic carrier protein coupled to a polymer, wherein the polymer comprises:
i) a first monomeric unit comprising a first monomer coupled to a hapten, and
ii) a second monomeric unit comprising a second monomer coupled to an immune cell receptor agonist.
   2. The conjugate of embodiment 1, wherein the polymer further comprises a third monomeric unit comprising a third monomer.
   3. The conjugate of embodiment 2, wherein the third monomeric unit does not comprise a hapten, drug, or biomolecule.
   4. The conjugate of embodiment 2, wherein the third monomeric unit comprises a molecular mass of less than about 250 Da, less than about 225 Da, less than about 200 Da, less than about 175 Da, less than about 150 Da, less than about 125 Da, or less than about 100 Da.
   5. The conjugate of embodiment 2, wherein the third monomeric unit is an acrylate, an acrylamide, a methacrylate, a methacrylamide, or a vinyl group.
   6. The conjugate of embodiment 5, wherein the third monomeric unit is hydroxyethyl methacrylamide.
   7. The conjugate of embodiment 1, wherein the first monomer is identical to the second monomer.
   8. The conjugate of embodiment 2, wherein the first, second, and third monomer are identical.
   9. The conjugate of embodiment 1, wherein the first monomer and the second monomer are each independently an acrylate, an acrylamide, a methacrylate, a methacrylamide, or a vinyl group.
   10. The conjugate of embodiment 1, wherein the first monomeric unit and the second monomeric unit are between about 1% and 50%, between about 1% and 5%, between about 1% and 10%, between about 1% and 15%, between about 1% and 20%, between about 1% and 25%, between about 1% and 30%, between about 1% and 40%, between about 2% and 5%, between about 2% and 10%, between about 2% and 15%, between about 2% and 20%, between about 2% and 25%, between about 2% and 30%, between about 2% and 40%, between about 2% and 50%, between about 5% and 10%, between about 5% and 15%, between about 5% and 20%, between about 5% and 25%, between about 5% and 30%, between about 5% and 40%, between about 5% and 50%, between about 10% and 20%, between about 10% and 30%, between about 10% and 40%, between about 10% and 50%, between about 15% and 25%, between about 15% and 30%, between about 15% and 40%, between about 15% and 50%, between about 20% and 30%, between about 20% and 40%, between about 20% and 50%, between about 25% and 40%, between about 25% and 50%, between about 30% and 40%, or between about 30% and 50% of monomeric units of the polymer.
   11. The conjugate of embodiment 1, wherein the first monomeric unit is between about 0.5% and 2.5%, between about 0.5% and 5%, between about 0.5% and 7.5%, between about 0.5% and 10%, between about 0.5% and 15%, between about 0.5% and 20%, between about 0.5% and 25%, between about 1% and 2.5%, between about 1% and 5%, between about 1% and 7.5%, between about 1% and 10%, between about 1% and 15%, between about 1% and 20%, between about 1% and 25%, between about 2.5% and 5%, between about 2.5% and 7.5%, between about 2.5% and 10%, between about 2.5% and 15%, between about 2.5% and 20%, between about 2.5% and 25%, between about 5% and 7.5%, between about 5% and 10%, between about 5% and 15%, between about 5% and 20%, between about 5% and 25%, between about 7.5% and 10%, between about 7.5% and 15%, between about 7.5% and 20%, between about 7.5% and 25%, between about 10% and 15%, between about 10% and 20%, between about 10% and 25%, or between about 15% and 25% of monomeric units of the polymer.
   12. The conjugate of embodiment 11, wherein the first monomeric unit is about 9% of the monomeric units of the polymer.
   13. The conjugate of embodiment 1, wherein the second monomeric unit is between about 0.5% and 2.5%, between about 0.5% and 5%, between about 0.5% and 7.5%, between about 0.5% and 10%, between about 0.5% and 15%, between about 0.5% and 20%, between about 0.5% and 25%, between about 1% and 2.5%, between about 1% and 5%, between about 1% and 7.5%, between about 1% and 10%, between about 1% and 15%, between about 1% and 20%, between about 1% and 25%, between about 2.5% and 5%, between about 2.5% and 7.5%, between about 2.5% and 10%, between about 2.5% and 15%, between about 2.5% and 20%, between about 2.5% and 25%, between about 5% and 7.5%, between about 5% and 10%, between about 5% and 15%, between about 5% and 20%, between about 5% and 25%, between about 7.5% and 10%, between about 7.5% and 15%, between about 7.5% and 20%, between about 7.5% and 25%, between about 10% and 15%, between about 10% and 20%, between about 10% and 25%, or between about 15% and 25% of monomeric units of the polymer.
   14. The conjugate of embodiment 13, wherein the second monomeric unit is about 15% of the monomeric units of the polymer.
   15. The conjugate of embodiment 2, wherein the first, second, and third monomeric units are between about 50% and 80%, between about 50% and 90%, between about 50% and 100%, between about 60% and 80%, between about 60% and 90%, between about 60% and 100%, between about 70% and 80%, between about 70% and 90%, between about 70% and 100%, between about 80% and 90%, between about 80% and 100%, between about 90% and 100%, between about 95% and 100%, about 100%, or 100% of the monomeric units of the polymer.
   16. The conjugate of embodiment 1, wherein the polymer is a random copolymer.
   17. The conjugate of embodiment 1, wherein the first monomer and the second monomer are non-peptidic.
   18. The conjugate of embodiment 1, wherein the polymer comprises a molecular weight of between about 0.1 to 200 kDa, between about 0.1 to 0.5 kDa, between about 0.1 to 1 kDa, between about 0.5 to 5 kDa, between about 0.5 to 10 kDa, between about 1 and 5 kDa, between about 1 and 10 kDa, between about 1 and 20 kDa, between about 1 and 30 kDa, between about 1 and 40 kDa, between about 1 to 50 kDa, between about 2 and 5 kDa, between about 2 and 10 kDa, between about 2 and 20 kDa, between about 2 and 30 kDa, between about 2 and 40 kDa, between about 2 and 50 kDa, between about 5 and 10 kDa, between about 5 and 20 kDa, between about 5 and 30 kDa, between about 5 and 40 kDa, between about 5 and 50 kDa, between about 5 and 100 kDa, between about 10 and 20 kDa, between about 10 and 30 kDa, between about 10 and 40 kDa, between about 10 and 50 kDa, between about 10 and 100 kDa, between about 10 and 200 kDa, between about 20 and 30 kDa, between about 20 and 40 kDa, between about 20 and 50 kDa, between about 20 and 100 kDa, between about 20 and 150 kDa, between about 20 and 200 kDa, between about 30 and 40 kDa, between about 30 and 50 kDa, between about 30 and 100 kDa, between about 30 and 150 kDa, between about 30 and 200 kDa, between about 50 and 100 kDa, between about 50 and 150 kDa, between about 50 and 200 kDa, between about 100 and 150 kDa, or between about 100 and 200 kDa.
   19. The conjugate of embodiment 18, wherein the polymer comprises a molecular weight of about 24 kDa.
   20. The conjugate of embodiment 1, wherein the polymer comprises between about 10 and 5000 monomeric units.
   21. The conjugate of embodiment 1, wherein the hapten is an opioid.
   22. The conjugate of embodiment 21, wherein the opioid is fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, or pharmacologically acceptable salt or solvate thereof.
   23. The conjugate of embodiment 22, wherein the opioid is fentanyl, a derivative thereof, a precursor thereof, or pharmacologically acceptable salt or solvate thereof.
   24. The conjugate of embodiment 23, wherein the opioid is fentanyl.
   25. The conjugate of embodiment 1, wherein the hapten is a chemical warfare agent.
   26. The conjugate of embodiment 25, wherein the chemical warfare agent is agent orange, 3-quinuclinidyl benzilate, VX gas, VR gas, HN1, HN2, or Tabun.
   27. The conjugate of embodiment 1, wherein the hapten is nicotine, cocaine, or MDMA.
   28. The conjugate of embodiment 1, wherein the hapten is a peptide or sugar derived from an infectious agent.
   29. The conjugate of embodiment 1, wherein the hapten is irreversibly coupled to the first monomer.
   30. The conjugate of embodiment 1, wherein the immune cell receptor agonist is an agonist of an intracellular receptor.
   31. The conjugate of embodiment 1, wherein the immune cell receptor agonist is a toll-like receptor agonist.
   32. The conjugate of embodiment 31, wherein the immune cell receptor agonist is a toll-like receptor 7 (TLR7) agonist, a toll-like receptor 8 agonist (TLR8), or an agonist of TLR7 and TLR8.
   33. The conjugate of embodiment 32, wherein the immune cell receptor agonist is a TLR7 agonist.
   34. The conjugate of embodiment 32, wherein the TLR7 agonist is imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, S-34240, SBT-6050, SBT-6290, ZM-TLR8, VX-001, MBS-8, APR-002, or a combination thereof.
   35. The conjugate of embodiment 32, wherein the TLR7 agonist is an imidazoquinoline.
   36. The conjugate of embodiment 35, wherein the TLR7 agonist is

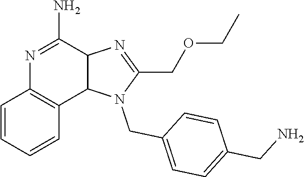 [SMILES: CCOCC1=NC2C(N)=Nc3ccccc3C2N1Cc1ccc(CN)cc1]

37. The conjugate of embodiment 1, wherein the immune cell receptor agonist is coupled to the second monomer by a cleavable linker.
38. The conjugate of embodiment 37, wherein the cleavable linker comprises a carbamate.
39. The conjugate of embodiment 1, wherein the immunogenic carrier protein is a pathogenic protein.
40. The conjugate of embodiment 1, wherein the immunogenic carrier protein is a bacterial protein.
41. The conjugate of embodiment 40, wherein the immunogenic carrier protein is a tetanus protein.
42. The conjugate of embodiment 41, wherein the immunogenic carrier protein is tetanus toxin.
43. The conjugate of embodiment 40, wherein the immunogenic carrier protein is a diphtheria toxin.
44. The conjugate of embodiment 43, wherein the immunogenic carrier protein is a genetically detoxified diphtheria toxin.
45. The conjugate of embodiment 44, wherein the immunogenic carrier protein is CRM197 (CRM).
46. The conjugate of embodiment 1, wherein the immunogenic carrier protein is coupled to the polymer by a cleavable linker.
47. The conjugate of embodiment 43, wherein the cleavable linker is a self-immolating linker.
48. The conjugate of embodiment 43, wherein cleavage of the cleavable linker releases the immunogenic carrier protein in an unmodified form.
49. The conjugate of embodiment 1, wherein the conjugate is water-soluble.
50. The conjugate of embodiment 1, wherein the conjugate is non-toxic.
51. The conjugate of embodiment 1, wherein the conjugate is configured to internalize within a B cell.
52. The conjugate of embodiment 48, wherein the B cell is an opioid-specific B cell.
53. A pharmaceutical composition comprising the conjugate of any one of embodiments 1-52 and a pharmaceutically acceptable excipient.
54. A method of treating addiction in a subject in need thereof comprising administering the conjugate of any of embodiments 1-52 or the composition of embodiment 49 to the subject, thereby treating the addiction in the subject.
55. The method of embodiment 54, wherein the addiction is an opioid addiction.
56. A method of preventing drug overdose in a subject in need thereof comprising administering the conjugate of any of embodiments 1-52 or the composition of embodiment 53 to the subject, thereby preventing the overdose in the subject.
57. The method of embodiment 56, wherein the overdose is an opioid overdose.
58. A vaccine composition comprising a conjugate comprising an immunogenic carrier protein coupled to a polymer, wherein the polymer comprises:
i) a first monomeric unit comprising a first monomer coupled to a hapten,
ii) a second monomeric unit comprising a second monomer coupled to an immune cell receptor agonist, and
   a third monomeric unit comprising a third monomer.
   59. The vaccine composition of embodiment 58, wherein the hapten is an opioid selected from the group consisting of fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, and pharmacologically acceptable salt or solvate thereof.
   60. The vaccine composition of embodiment 58, wherein the immune cell receptor agonist is a toll-like receptor (TLR) agonist selected from the group consisting of imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, S-34240, SBT-6050, SBT-6290, ZM-TLR8, VX-001, MBS-8, APR-002, imidazoquinoline,

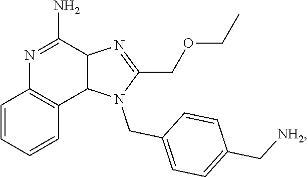 [SMILES: CCOCC1=NC2C(N)=Nc3ccccc3C2N1Cc1ccc(CN)cc1]

and fragment thereof.
61. The vaccine composition of embodiment 58, wherein the immunogenic carrier protein is selected from the group consisting of tetanus toxins, diphtheria toxins, related proteins such as CRM197, genetically toxoided variant of diphtheria toxin, meningococcal outer membrane protein complex (OMPC), H. influenzae protein D (HiD), Keyhole Limpet Hemocyanin (KLH), Bovine serum albumin (BSA), Ovalbumin (OVA), and recombinant P. aeruginosa exoprotein.
62. A vaccine composition comprising a conjugate comprising ovalbumin coupled to a polymer, wherein the polymer comprises:
i) a first monomeric unit comprising a first monomer coupled to fentanyl,
ii) a second monomeric unit comprising a second monomer coupled to a TLR7 agonist, a TLR8 agonist, or a TLR7/TLR8 agonist, and
iii) a third monomeric unit comprising a third monomer.
   63. A vaccine composition comprising a conjugate comprising CRM197 coupled to a polymer, wherein the polymer comprises:
i) a first monomeric unit comprising a first monomer coupled to fentanyl,
ii) a second monomeric unit comprising a second monomer coupled to a TLR7 agonist, a TLR8 agonist, or a TLR7/TLR8 agonist, and
iii) a third monomeric unit comprising a third monomer.
   64. A vaccine composition comprising a conjugate comprising KLH coupled to a polymer, wherein the polymer comprises:
i) a first monomeric unit comprising a first monomer coupled to fentanyl,
ii) a second monomeric unit comprising a second monomer coupled to a TLR7 agonist, a TLR8 agonist, or a TLR7/TLR8 agonist, and
iii) a third monomeric unit comprising a third monomer.
   65. A method of vaccinating a subject in need thereof against substance abuse comprising administering to the subject a vaccine composition comprising an immunogenic carrier protein coupled to a polymer, wherein the polymer comprises:
i) a first monomeric unit comprising a first monomer coupled to a hapten,
ii) a second monomeric unit comprising a second monomer coupled to an immune cell receptor agonist, and
iii) a third monomeric unit comprising a third monomer.
   thereby vaccinating the subject against substance abuse.
   66. The method of embodiment 65, wherein the immunogenic carrier protein coupled to a polymer is OVA, the hapten is fentanyl, and the immune cell receptor agonist is a TLR7 agonist, a TLR8 agonist, or a TLR7/TLR8 agonist.
   67. The method of embodiment 65, wherein the immunogenic carrier protein coupled to a polymer is CRM197, the hapten is fentanyl, and the immune cell receptor agonist is a TLR7 agonist, a TLR8 agonist, or a TLR7/TLR8 agonist.
   68. The method of embodiment 65, wherein the immunogenic carrier protein coupled to a polymer is KLH, the hapten is fentanyl, and the immune cell receptor agonist is a TLR7 agonist, a TLR8 agonist, or a TLR7/TLR8 agonist.
   69. A method of preventing drug overdose in a subject in need thereof comprising administering to the subject a conjugate comprising an immunogenic carrier protein coupled to a polymer, wherein the polymer comprises:
i) a first monomeric unit comprising a first monomer coupled to a hapten, and
iv) a second monomeric unit comprising a second monomer coupled to an immune cell receptor agonist,
   thereby preventing drug overdose in the subject.
   70. The method of embodiment 69, wherein the immunogenic carrier protein coupled to a polymer is OVA, the hapten is fentanyl, and the immune cell receptor agonist is a TLR7 agonist, a TLR8 agonist, or a TLR7/TLR8 agonist.
   71. The method of embodiment 69, wherein the immunogenic carrier protein coupled to a polymer is CRM197, the hapten is fentanyl, and the immune cell receptor agonist is a TLR7 agonist, a TLR8 agonist, or a TLR7/TLR8 agonist.
   72. The method of embodiment 69, wherein the immunogenic carrier protein coupled to a polymer is KLH, the hapten is fentanyl, and the immune cell receptor agonist is a TLR7 agonist, a TLR8 agonist, or a TLR7/TLR8 agonist.

The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Vaccine Synthesis

This example covers the synthesis of a polymeric fentanyl vaccine using block copolymers, termed p(TLR7/8-b-Fent), bearing ligands for toll-like receptors 7 & 8 (TLR7/8) (i.e., R848) and fentanyl. This conjugate p(TLR7/8-b-Fent) was then coupled to the carrier protein ovalbumin (OVA) (FIG. 2).

p(TLR7/8-b-Fent) was synthesized via a reversible addition fragmentation chain transfer (RAFT) polymerization using an azido-functionalized RAFT agent, a biologically inert co-monomer, and methacrylamide-modified TLR7/8 and fentanyl monomers (FIG. 2). A p(Fent) polymer without TLR7 agonist was separately synthesized using the same synthetic approach (FIG. 3). Next, OVA was modified with a di-functional polyethylene glycol linker that forms a self-immolative linkage upon protein conjugation and endows OVA with azido-reactive handles for p(Fent-b-TLR7) conjugation (FIG. 2). The self-immolative linkage is stable prior to cell-internalization, where it cleaves releasing the protein in its unmodified form. Finally, both p(TLR7/8-b-Fent) and p(Fent) were conjugated to linker-modified OVA to form OVA-p(TLR7/8-b-Fent) and OVA-p(Fent). Additionally, a monomeric fentanyl vaccine was synthesized (OVA-mFent) by reacting azido-modified fentanyl moieties to linker-modified OVA to produce (FIG. 2). All polymer and monomer structures were verified via NMR and polymer and protein conjugates were characterized via size exclusion chromatography.

Using this synthetic strategy, p(TLR7/8-b-Fent) was synthesized. The conjugate included both fentanyl monomer (˜2.5 mol %) and TLR7/8 monomer (˜4.5 mol %) in a water-soluble block co-polymer. This co-polymer was conjugated to OVA to generate OVA-p(TLR7/8-b-Fent) with 85% yield.

### Example 2

### Vaccine-Induced Immune Responses

This example covers opioid vaccination with the polymeric fentanyl vaccine of EXAMPLE 1. It was hypothesized herein that a polymeric conjugate vaccine composed of a carrier protein modified with polymers decorated with opioids and a B cell-activating adjuvant would enhance BCR crosslinking and localize immune stimulation to B cells, resulting in enhanced B cell activation and anti-opioid Ab responses.

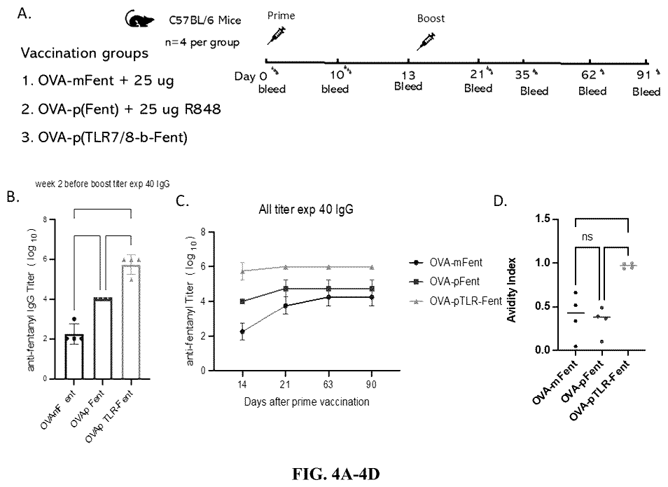

On day 0 and 14, C57BL/6 mice (n=4 per group) were vaccinated subcutaneously with OVA-p(TLR7/8-b-Fent) or TLR7/8 admixed with OVA-p(Fent) or OVA-mFent and then bled via facial vein sampling at the times shown in FIG. 4A.

Fentanyl specific serum IgG antibody response was measured by standard ELISA procedure using fentanyl conjugated BSA (10 ng/well) as capture antigen. Endpoint titers were determined using Titer=Blank mean+4*SD of blank. ELISAs against BSA were used to verify fentanyl-specificity. Avidity of anti-fentanyl serum IgG was determined by chaotropic ELISA. The procedure followed the same steps as above with the addition of incubation with 1M Guanidine after serum binding. Avidity was determined by calculating the ratio between the Absorbance450 with and without a chaotropic agent.

It was demonstrated that OVA-p(TLR7/8-b-Fent) offers an improved anti-fentanyl IgG antibody response with superior avidity against fentanyl than a state-of-the-art monomeric vaccine (FIG. 4). Given the modularity of our approach we anticipate that our polymeric vaccine can be easily adapted to protect against numerous other opioids and infectious diseases.

Vaccination with OVA-p(TLR7-b-Fent) induced a more superior fentanyl-specific IgG response than vaccination with our TLR7/8 monomer admixed with OVA-p(Fent) or OVA-mFent. Thus, the presence of TLR7/8 agonist in the polymer backbone and multivalent display of fentanyl on the polymer have a profound influence on the humoral immune response toward fentanyl, suggesting elevated B cell receptor (BCR) crosslinking and subsequent TLR7/8-mediated B cell activation. OVA-p(TLR7/8-b-Fent) induced 1000-fold increase in anti-fentanyl antibody endpoint titer as compared to monomeric OVA-mFent over 90 days (FIG. 4B-4C). No cross reactivity was observed with BSA coated plate suggesting antibody response is Fentanyl specific (data not shown). In addition, OVA-p(TLR7/8-b-Fent) elicited antibodies that bind fentanyl with greater avidity than the antibody repertoire generated by the TLR7/8 monomer admixed with OVA-p(Fent) or OVA-mFent (FIG. 4D).

### Example 3

### Vaccine-Induced Immune Responses with a CRM197-Conjugate

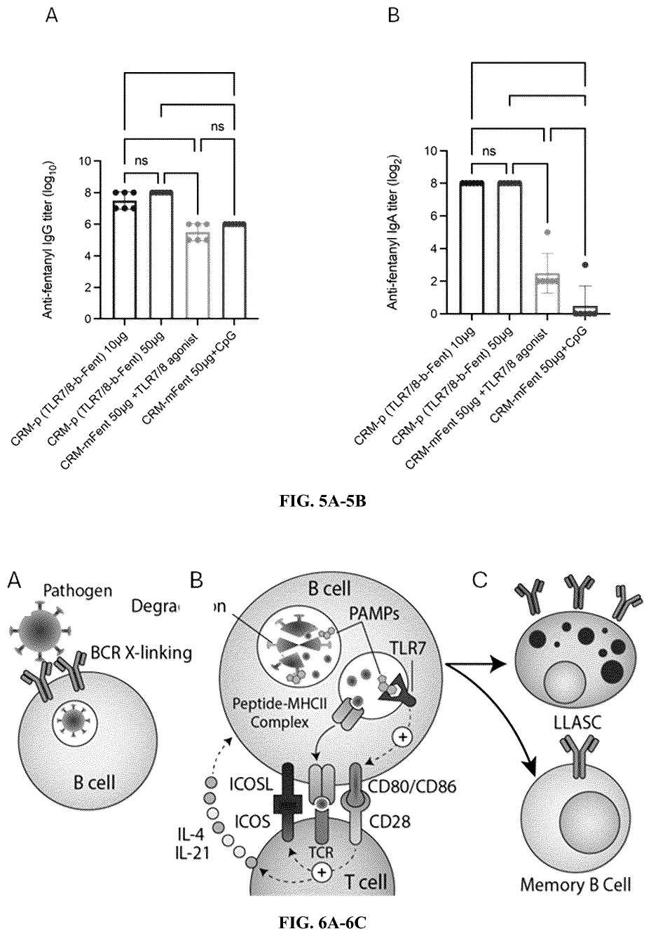

This example is directed to opioid vaccination with a conjugate of CRM197. CRM197 (CRM) is a genetically detoxified diphtheria toxin that can be used as a carrier protein in conjugate vaccines. On day 0 and 14, C57BL/6 mice (n=6 per group) were vaccinated subcutaneously with CRM-p(TLR7/8-b-Fent) (10 mg or 50 mg) or TLR7/8 admixed with CRM-mFent (50 mg) or CRM-mFent (50 mg) admixed with CpG and then bled via facial vein sampling on day 21. The serum pan anti-fentanyl IgG and IgA titer were determined via ELISA (FIG. 5).

At an equivalent dose of 50 mg of vaccine, CRM-p(TLR7/8-b-Fent) induces a more robust anti-fentanyl IgG titer than CRM-mFent admixed with TLR7 or CpG. Even at a dose of 10 mg, CRM-p(TLR7/8-b-Fent) induces a greater magnitude anti-fentanyl IgG tier than the CRM-mFent vaccines tested here at 50 mg (FIG. 5A). In addition, CRM-p(TNR7/8-b-Fent) at a dose of 10 mg and 50 mg induces a greater anti-fentanyl IgA titer than the CRM-mFent formulations with TLR7/8 agonist or CpG (FIG. 5B).

### Example 4

### Rational and Mechanism of Action of the Vaccine of the Invention

Durable antibody-mediated immune responses (i.e., humoral immunity) are maintained by LLASCs and memory B cells. Once generated, LLASCs continuously produce antibodies and can persist in the body for decades and provide lasting immunosurveillance. Although memory B cells do not produce antibody, they can persist for a lifetime in the body and, upon reencounter of their cognate antigen during subsequent vaccinations, differentiate into LLASCs that produce antibodies with a greater affinity than the parent memory B cell.

LLASCs and memory B cells are generated in the secondary lymphoid organs as a consequence of B cell activation in the presence of T cell help, a process termed T cell-depend B cell activation (FIG. 6A-6C). T cell-dependent B cell activation requires two distinct signals: (1) BCR recognition of its cognate antigen, and (2) recognition of T cell-derived costimulatory signals. In the context of humoral responses to invading pathogens, the first activation signal occurs upon BCR (i.e., a membrane-bound antibody) recognition of its cognate antigen, resulting in B cell activation and the expression of membrane bound proteins (e.g., CD86, CD80) and soluble cytokines (e.g., IL-6) that activate helper T cells (FIG. 6A). BCR crosslinking by multivalent proteins (i.e., antigens) on the surface of invading pathogens causes BCRs to internalize the pathogen by receptor-mediated endocytosis. Upon internalization, the pathogen's antigens are digested into their constituent peptides, which are complexed with MHCII molecules (FIG. 6B). These MHCII-peptide complexes are trafficked to the surface of the B cells, enabling the B cell to present antigen to CD4⁺ T cells. T cell receptors (TCR) on pathogen-specific T helper cells bind to the pathogen peptide-MHC II complex and ligate activated B cell-derived costimulatory signals resulting in T helper cell activation. Activated T cells then express B-cell activating proteins (e.g., IL-4, IL-21, ICOS) that act as the second signal necessary to drive B cells to differentiate into memory B cells and LLASCs (FIG. 6C). In addition to these two necessary signals, B cell recognition of pathogen-associated molecular patterns (PAMPs) by B cell pattern recognition receptors (PRRs), such as TLR7 (FIG. 6B), results in enhanced B cell activation, increasing the induction of memory B cells and LLASCs.

### Example 5

### Synthesis of TT-p(Fent-co-TLR7)

To demonstrate that polymeric fentanyl conjugate vaccine induces robust α-fentanyl immune responses, TT-p(Fent-co-TLR7) was synthesized, and the immunogenicity of TT-p(Fent-co-TLR7) was compared to a monomeric fentanyl conjugate vaccine in mice.

The p(Fent-coTLR7) used in this disclosure was synthesized via the reversible addition fragmentation chain transfer (RAFT) polymerization strategy shown in FIG. 7. The polymer was synthesized via RAFT polymerization due to RAFT's ability to produce polymers with predefined weights and low polydispersity from monomers bearing a wide array of functional units. Furthermore, RAFT has previously been utilized to synthesize biomaterial-based drug delivery strategies for the treatment of malaria, cranial re-synostosis, autoimmunity, and osteoarthritis. In order to generate a polymeric conjugate fentanyl vaccine p(Fent-co-TLR7) was synthesized from an azide-terminated RAFT agent 1, a methacrylamide-modified TLR7-ligand 2 (mTLR7), a methacrylamide-modified fentanyl analogue 3 (fentanyl-monomer), and the biologically inert co-monomer N-(2hydroxyethyl)methacrylamide 4 (HEMA). Compounds 1 and 2 were synthesized via previously reported procedure and the fentanyl-monomer was synthesized by modifying a reported procedure for the synthesis of fentanyl analogs. The composition and molecular weight of the p(Fent-co-TLR7) produced for our preliminary studies was confirmed via ¹H NMR, C₁₃NMR, and gel permeation chromatography (GPC). The molar ratio of mTLR7 and fentanyl-monomer in our p(Fent-co-TLR7) was 0.15 and 0.09 respectively and the weight average molecular weight was ˜24 kDa.

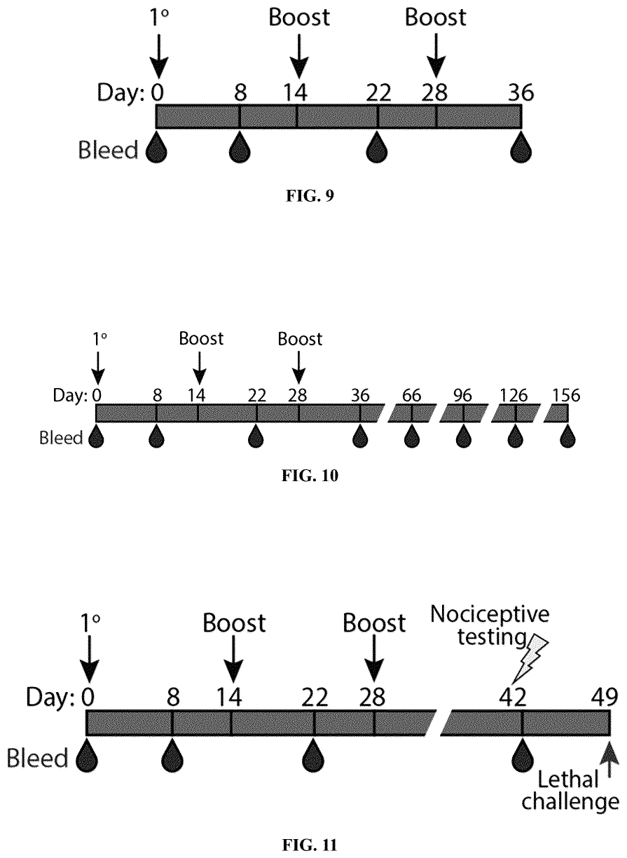

TT was then conjugated to p(Fent-co-TLR7) via the synthetic pathway shown in FIG. 8. To conjugate p(Fent-co-TLR7) to TT, we synthesized a bifunctional poly(ethylene glycol) linker 7 functionalized with an amine-reactive NHS-carbonate and bicyclononyne (BCN)—a copper-free click ligand that spontaneously reacts with azides—via our previously reported protocol¹⁴. TT was then conjugated to 7 to generate TT-PEG-BCN 8, which was isolated via SEC. Upon conjugation, the heterobifunctional linker forms a disulfide-reduction-sensitive self-immolative linkage that is known to be relatively stable in serum but cleaves upon endocytosis (FIG. 8). TT-PEG-BCN was then mixed with excess p(Fent-co-BCN) in PBS at room temperature to from TT-p(Fent-co-TLR7) 9, which was purified via SEC and the molecular weight of the conjugates was verified via SDS gel electrophoresis (data not shown). According to SDS page, our TT-p(fent-co-TLR7) conjugates were composed of ˜3-4 polymer chains per TT.

### Example 6

### Optimization of Formulation and Mechanisms Responsible for the Efficacy of TT-p(Fent-Co-TLR7)

Described herein is the synthesis of a library of polymers to test the effect of the polymer's monomer content and molecular weight on the immunogenicity of TT-p(Fent-co-TLR7). Disclosed herein is an optimized TT-p(Fent-co-TLR) formulation, with which animal studies can be performed to compare the efficacy of TT-p(Fent-co-TLR7) to constituent formulations lacking the functionalities of our vaccine platform. Described herein is a biodistribution study to measure the ability of TT-p(Fent-co-TLR7) to target and activate B cells.

Disclosed herein is the synthesis of TT-p(Fent-co-TLR7) conjugates. Although the p(Fent-co-TLR7) generated for preliminary studies generated a robust α-fentanyl response, the molar ratio of monomers that comprise the polymer and the polymer's molecular weight should affect the immunogenicity of TT-p(Fent-co-TLR7) conjugates. Described herein are the effects of polymer monomer content and molecular weight on the vaccine and an optimized TT-p(Fent-co-TLR7) formulation. A library of p(Fent-co-TLR7) polymers can be synthesized and characterized according to the protocol described in above. In some aspects, the synthesized polymers will have the monomer mole fractions and degrees of polymerization (DP) shown in Table 1. In a particular aspect, each of the polymers contain a 0.15 mole fraction of mTLR7 based on previous studies showing that a 0.15 mole fraction of mTLR7 in mTLR7-containing polymers was optimal for activating both murine and human TLR7.

In some aspects, TT can then be modified with the bifunctional linker via the protocol described above and shown in FIG. 8 so that each TT is modified with an average of 4 PEG linkers per TT. The number of polymers conjugated to TT is limited given that it was shown that TT-p(Fent-co-TLR7) with more than 4 polymer units are prone to aggregation. Finally, TT-p(Fent-co-TLR7) conjugates can be made with polymers 1-4 in Table 1 and purified via SEC. The molecular weight of the TT-p(Fent-co-TLR7) conjugates can be verified via gel electrophoresis, MALDI, and GPC.

Described in the present disclosure is immunogenicity of TT-p(Fent-co-TLR7) conjugates. An optimal TT-p(Fent-co-TLR7) formulation can be determined by vaccinating BALB/c mice (n=7/group) via subcutaneous (s.c.) injection into the four footpads with saline or 20 μg of TT formulated as a TT-p(Fent-co-TLR7) conjugate vaccine synthesized from one of the polymers listed in Table 1. Mice are to be treated and bled according to the timeline in FIG. 9. Total α-fentanyl IgG titer can be measured via fentanyl-specific ELISA to determine the kinetics of the α-fentanyl response. The formulation that produces the greatest α-fentanyl IgG response on day 36 will be selected as our lead formulation and used for subsequent studies.

Described in the present disclosure a way to measuring the α-fentanyl response via ELISA. The α-fentanyl-specific IgG antibody titers can be determined by ELISAs using plates coated with fentanyl-bovine serum albumin (BSA) conjugates (Creative Biolabs). Given that the mice are not vaccinated with BSA, the measured titer to fentanyl-BSA should be specific to fentanyl. However, to confirm the fentanyl-specificity of the response, the absence of an α-BSA titer in the serum samples can be confirmed via BSA ELISA. Importantly, to exclude the confounding effects of pre-existing immunity to BSA, only mice lacking a measurable α-BSA titer on day zero can be included in the study.

Described in the present disclosure is the functionality of TT-p(Fent-co-TLR7)'s design elements. Disclosed herein is TT-p(Fent-co-TLR7)'s ability to target and crosslink fentanyl specific BCRs, activate TLR7, and release unmodified TT as integral to its functionality. To demonstrate that the individual design elements contribute to the efficacy of TT-p(Fent-co-TLR7) conjugates and that the “whole” of TT-p(Fent-co-TLR7) is more than just “the sum of its parts”, BALB/c mice (n=7/group) can be vaccinated via subcutaneous injection into the four footpads with saline, 20 μg of TT formulated as TT-p(Fent-TLR7) or one of the constituent vaccines shown in Table 2, which each lack one of the functionalities of TT-p(Fent-TLR7). The mice will be vaccinated and bled according to the schedule in FIG. 4. The α-fentanyl response in the serum will be assessed as described in Section 4.1.2. Additionally, the mice will be sacrificed on day 36 and the fentanyl-specific B cell and TT-specific CD4⁺ T cell responses in the secondary lymphoid organs will be characterized.

TABLE 2
Control polymers lacking a functionality of TT-p(Fent-co-TLR7)

| | Conjugates | | Missing |
|---|---|---|---|
| Name | made without | Substitution | Functionality |
| TT-p(Frat) + | TLR7-monomer | HEMA | Co-delivery of |
| mIIR7 | | | polymeric |
| | | | TLR7 |
| TT-p(TLR7) + | Fentanyl- | HEMA | Fentenyl-BCR |
| mFent | monomer | | Targeting |
| TT-Traut- | Self-Immolative | p(Fenboo-1121.7) | Release of |
| p(Fent-co-TLR7) | linkage | conjugated via. | unmodifed TT |
| | | Traits Reagent | |
| IT + p(Fent-co- | Conjugation of | Admixture | Delivery of |
| TLR7) | p(lent-co- | offland p(Fent- | Tito B cells |
| | Eit7)tolT | co-RR7) | |

Disclosed herein is Fentanyl-specific B cell response. Spleen, dLNs (i.e., inguinal, popliteal, axial, brachial), and bone marrow (BM) can be harvested, and the resident cells can be isolated via physical and enzymatic disruption then washed via centrifugation. Cell populations will then be stained with fluorescently-labeled antibodies for phenotypic markers delineating ASC, LLASCs, and memory B cells. Cells can also be stained with fluorescently-labeled BSA and fentanyl-BSA conjugates to identify fentanyl-specific cell populations (Table 3). The percentage and total number of fentanyl-specific ASCs, LLASCs, and memory B cells will be determined via flow cytometry.

TABLE 3
Identification of fentanyl-specific B
cells, memory B cells, ASCs, and LLASCs

| Cell Type | Phenotypic Markers |
|---|---|
| Antibody Secreting Cell (Le., | CD3−, CD1 lb−, CD19+, |
| Plasma cells) | B220(lo), CD138+ |
| Long-lived ASCs (Plasma cells, | CD3−, CD1 lb−, CD19+, |
| BM) | B220(1o), CD138+, CD44+, CD93+ |
| Memory B cells | B220(high), CD19+, CD273+, |
| | CD38+, GL7−, CD138− |
| Antigen Specificity | Phenotypic Markers and |
| | (Fentanyl-BSA)-F, BSA− |

Described in the present disclosure is TT-specific CD4⁺ T cell response. By targeting and crosslinking fentanyl-specific BCRs and releasing unmodified TT, described herein is TT-p(Fent-co-TLR7) designed to optimize TT-specific T cell activation to maximize T cell help to fentanyl-specific B cells. To measure the TT-specific CD4⁺ T cell response, total cell populations from the spleen and dLNs can be re-exposed to TT in vitro. TT re-exposure reactivates TT-specific T cells allowing to measure their activity and phenotype. After 6 h of antigen-specific restimulation, the cells can be stained with fluorescently-labeled antibodies for CD4⁺ and CD8⁺ T cell-specific surface markers and for the intra-cellular cytokines IFN-7, TNF-α, IL-2, and IL-4. The percentage and total number of cytokine-producing (i.e., activated) CD4⁺ T cells can be evaluated by flow cytometry. Additionally, using flow cytometry the expansion of T follicular helper (Tfh) cells (CD4⁺ PD-1⁺CXCR5⁺CD44⁺ICOS⁺)—specialized T cells that assist in B cell development can be quantified. Cell populations from the spleen and draining dLNs can also be restimulated with TT for 72h and the concentration of IFN-7, TNF-α, IL-2, and IL-4 in the cell media can be measured via sandwich ELISA.

Described herein is biodistribution of TT-p(Fent-co-TLR7). The efficacy of TT-p(Fent-co-TLR7) is predicated on its ability to target and activate fentanyl-specific B cells. To demonstrate the targeting and B cell activating prowess of TT-p(Fent-co-TLR7) BALB/c can be vaccinated via subcutaneous injection into the four footpads with saline, polymeric conjugates labeled with Alexa Fluor™ 647 (TT₆₄₇-p(Fent-co-TLR7)), or Alexa Fluor™ 647-labeled monomeric fentanyl vaccine conjugates (TT₆₄₇-mFent) and CpG. After 12 h, the spleen and dLNs can be harvested and the resident cells will be isolated. The spleen and dLN cell populations can be stained with fluorescently-labeled antibodies to identify B cells, T cells, classical dendritic cells (cDCs), plasmacytoid DCs (pDCs), and macrophages and their activation state (Table 4). Fentanyl-specific B cells can be labeled with fluorescently-labeled fentanyl-BSA conjugates and BSA labeled with a different fluorophore and identified as fentanyl-BSA⁺BSA⁻. Flow cytometry can be used to identify the percentage and number of each immune cell population positive for TT₆₄₇-p(Fent-co-TLR7) or TT₆₄₇-mFent. In some aspects, the activation state of B cells and APCs that stain positive for vaccine uptake can be determined by the expression of relevant activation markers (Table 4).

TABLE 4
Identification of activated mouse immune cells

| Cell Type | Phenotypic Ala ref s |
|---|---|
| CD3+ T cells | O34− O33+ O38+ B220− CDl 1c−, |
| | F4/8 0− |
| CD4+ T cells | O34+ O33+ O38− B220− CDl 1c−, |
| | F4/80− |
| B cells | CD19+, B220+, CD3−, CD11c−, |
| | CD11b−, |
| CD3− cDC | CD19− N K1.1− CD3− MHCII+ |
| | CD11c+ CD11b+ CD8− |
| C D8+ cDC | W19− N K1.1− CD3− MHCII+ |
| | CD11c+ CD11b(l 01 CD8+ |
| CD103+ cDC | W19− N K1.1− CD3− MHCII+ |
| | CD11c+ CD11b(l 01 CD103+ |
| pDC | W19− N K1.1− CD3− MHCII+ |
| | CD11c+ CD11b(l 01 B220+ |
| Macrophage | CD19− N K1.1− CD3− MHCII+ |
| | CD11c(I 01 CD11b+ F4/80+ |
| DC and macrophage activation | CD80/CD36/MHC II(hi) |
| markers | |
| B cell activation markers | CD80/CD86/CD69 |

Described herein are polymers designed to simultaneously crosslink BCRs and, upon internalization, activate endosomal TLR7, resulting in targeted B cell activation. Disclosed herein is increased uptake of TT-p(Fent-co-TLR7) by B cells and measurement of elevated levels of B cell activation marker expression by B cells that have taken up the vaccine.

### Example 7

### Comparing TT-p(Fent-Co-TLR7) to Monomeric Fentanyl Conjugate Vaccines

Described herein is the comparison of the immunogenicity of TT-p(Fent-co-TLR7) to TT-mFent admixed with free mTLR7 (the monomer used in p(Fent-co-TLR7)) or CpG-a TLR9 agonist commonly used in experimental opioid vaccines.

Described herein is TT-p(Fent-co-TLR7) vs. TT-mFent. In some aspects, BALB/c mice are treated with 20 μg of TT formulated as TT-p(Fent-co-TLR7), TT-mFent+mTLR7, or TT-mFent+CpG (CpG: 50 μg) via subcutaneous injection into the four footpads and bled according to the treatment schedule shown in FIG. 9. In a particular aspect, the amount of mTLR7 used to formulate TT-mFent+mTLR7 is equal to the amount contained in TT-p(Fent-co-TLR7). For example, the mice are monitored after treatment and weighed daily as a measure of toxicity. In some aspects, total α-fentanyl IgG in the serum samples is measured via fentanyl-specific ELISA. In further aspects, serum protein, bilirubin, urea, nitrogen, IL-12, TNF-α, and alanine aminotransferase activity is measured in the serum samples as indicators of toxicity. In one aspect, on day 36, the mice are sacrificed the fentanyl-specific B cell and TT-specific CD4⁺ T cell responses in the secondary lymphoid organs will be characterized as described herein.

Described herein is durability of α-fentanyl response. In some aspects, to elucidate the durability of the α-fentanyl IgG response and the induction of LLASCs, BALB/c mice are vaccinated with saline, or 20 μg of TT formulated as TT-p(Fent-co-TLR7), TT-mFent+mTLR7, or TT-mFent+CpG (CpG: 50 μg) via subcutaneous injection into the four footpads and the mice bled according to the treatment schedule shown in FIG. 10. In some aspects, total α-fentanyl IgG in the serum samples is measured via fentanyl-specific ELISA. In a particular aspect, on day 156 the mice are sacrificed and the relative quantity of fentanyl-specific LLASCs and memory B cells in the bone marrow and spleen, respectively, are enumerated via B cell ELISpot.

Described herein is B cell ELISpot. To evaluate the fentanyl-specific LLASCs and memory B cells, ELISPOT plates (Millipore) are coated with fentanyl-BSA conjugates or coated with anti-mouse IgG to quantify total LLASC number. Coated plates are washed with PBS and blocked in RPMI culture media (10% FBS) for 2 hours. For LLASCs quantification, cell suspensions prepared from the bone marrow are serially diluted in culture media and transferred to the blocked, coated ELISpot plates. For memory B cell quantification, cell suspensions prepared from the spleen are first be incubated in media supplemented with R848 and IL-2 for 5 days to activate memory B cells. The activated memory B cells are serially diluted in culture media and transferred to blocked, coated ELISpot plates. Following 24 h incubation at 37° C., the plates are washed and treated with biotinylated goat anti-mouse total IgG (Southern Biotech) 1.5 h. Plates are washed and treated with streptavidin alkaline phosphatase (Vector Labs) in PBS/Tween-20 for 1 h. Presence of fentanyl- or IgG-specific antibodies can be detected using NBT/BCIP colorimetric substrate (eBioscience). The spots, each indicative of an antibody secreting cell, can be counted using the CTL ImmunoSpot ELISPOT software.

Described herein, is the possibility of TT-p(Fent-co-TLR7) to generate a greater α-fentanyl IgG and TT-specific T cell response that the monomeric conjugate vaccines tested herein. Although TLR7 small-molecule agonists have been shown to induce systemic immune activation, by tethering the TLR7 ligand to a construct that targets BCRs, TT-p(Fent-co-TLR7) should localize TLR7 activation to B cells thus preventing an TLR7-mediated toxicity. Durable antibody responses are maintained by LLASCs, which are generated upon B cell activation in the presence of T cell help. TT-p(Fent-co-TLR7) is specifically designed to optimize T cell help by delivering unmodified TT and maximizing B cell-derived T cell co-stimulatory signals. Described herein is TT-p(Fent-co-TLR7) that can generate a long-lasting α-fentanyl response that correlates with LLASC and memory B cell numbers.

Described herein is a way to demonstrate protection from the antinociceptive and lethal effects of fentanyl. Given that the antinociceptive effects of fentanyl require the drug to pass into the central nervous system, the ability of our vaccine to prevent fentanyl diffusion into the brain can demonstrated by measuring the absence of antinociception after fentanyl challenge. In some aspects, BALB/c mice are vaccinated via subcutaneous vaccination in the four footpads with saline, or 20 μg of TT formulated as TT-p(Fent-co-TLR7) or TT-mFent+CpG (CpG: 50 μg) (n=12 mice/group) and the mice are bled according to FIG. 11. Total α-fentanyl IgG titers in the serum samples can be measured via fentanyl-specific ELISA. In some aspects, on day 42, the mice in each treatment group are randomly assigned to two sub-groups that undergo either hot plate or tail flick antinociceptive testing. In some aspects, on day 49, all mice are administered a lethal dose of fentanyl, and the survival of the animals are documented. Antinociceptive and survival test results are correlated with α-fentanyl IgG in the serum.

Described herein is antinociceptive testing. In some aspects, on day 42, mice are tested for cumulative fentanyl response in primarily supraspinal (hot plate) and spinal (tail flick) behavioral tests. For the hot pate test, the mice are habituated to the testing environment for 1h and weighed before being placed into an acrylic cylinder on a 54° C.±0.2 C hotplate for a maximum of 60 sec (i.e., “cutoff” time). In some aspects, the time required to achieve a nociceptive response of hind paw licking, shaking/withdrawal of hind paws, or jumping is recorded as the “baseline” latency to respond time. In some aspects, fentanyl is injected intraperitoneally at increasing doses of 0.01, 0.05, 0.25, 0.5, 0.75 mg/kg to generate a full dose-response curve. The nociceptive test can be repeated at 15 min intervals following each injection and the cycle of test and injection repeated with increasing cumulative dosing until full antinociception is observed.

For the tail flick measurements, the animals are allowed to habituate to lab surroundings for an hour before analysis and weighed before the start of the experiment. Mice can be lightly restrained in the tail flick apparatus (PNLAB 7160, Spain) and radiant heat applied to the tail 5-8 cm from the tip. The tail flick reaction time can be recorded as the “baseline” latency to response time. The cutoff time can be set to 15s to prevent tissue damage to the tail. The baseline latency response time can be recorded three times at different positions on the tail for each measurement. The mice can then be administered an intraperitoneal injection of fentanyl at increasing doses of 0.01, 0.05, 0.25, 0.5, 0.75 mg/kg and the “test” tail flick latency time recorded 15 min after each escalating does until full antinociception is observed.

Both hot plate and tail flick antinociception data can be transformed from time to percent maximum possible effect (% MPE) using the standard formula: % MPE=(test−baseline)/(cutoff−baseline)×100. The data can then be fit using a non-linear regression (log(dose) vs. % MPE) in GraphPad PRISM and the ED₅₀ values and 95% confidence intervals determined for each test and individual treatment group.

Described herein is Lethality Challenge. In some aspects, on day 49, all the mice in each group are administered a lethal dose of fentanyl (e.g., 5.0 mg/kg) via intraperitoneal injection and the survival of the animals are monitored over the following 4 h.

Described herein is a demonstration that TT-p(Fent-co-TLR7) generates a more robust α-fentanyl IgG response than TT-mFent, and that TT-p(Fent-co-TLR7) is more effective at preventing the antinociception and lethal effects of fentanyl as compared to TT-mFent+CpG.

### Example 8

### Polymeric Vaccine Outperforms Monomeric Vaccine

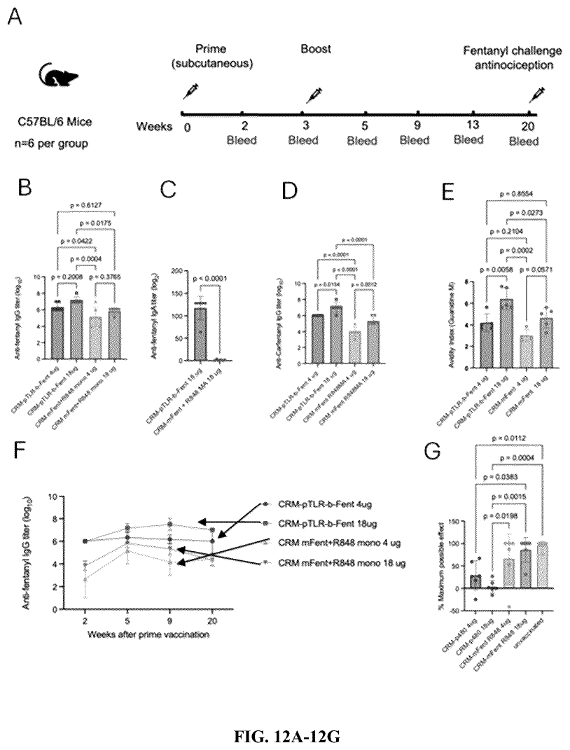

C57Bl/6 mice were vaccinated with CRM-pTLR-b-fent and CRM-mFent conjugate at two different doses 4 μg and 18 ug (of CRM and TLR 7 agonist weight equiv.) per mouse on day 0 and 21 (FIG. 12A). Mice were bled at different time points. Anti-fentanyl serum IgG (FIG. 12AB) and IgA (FIG. 12C) and anti-carfentanyl IgG (FIG. 12D) antibody level determined by ELISA on week 5. Avidity was measure using modified chaotropic ELISA using guanidine as denaturant. Avidity index (FIG. 12E) is amount of guanidine required to inhibit the 50% binding of serum antibody on the plate. Long term anti-fentanyl IgG responses were measured over 20 weeks (FIG. 12F). Nociception was measured on week 20 post vaccination using hot plate analgesia meter (Columbus instruments) post 30 min i.p. challenge of fentanyl HCl. (0.25 mg/kg). the baseline latency was measured prior to drug injection by observing one of the following nociception responses: hind paw licking or jumping with 60 s maximum cutoff time to prevent tissue damage. The percentage maximum possible effect (% MPE) (FIG. 12G) was calculated as the post-test latency minus the pretest latency divided by the maximum time (60 seconds) minus the pretest latency times 100. All statistical differences were determined by one-way ANOVA using Brown-Forsythe test where p<0.05 represent statistical significance with respect to all other treatment groups or as indicated. The end-point titers were defined as the highest dilution at which the 450 nm OD value was above the mean+4 SD of that of sample with no serum.

### Example 9

### Long Term Anti-Fentanyl IgG Responses

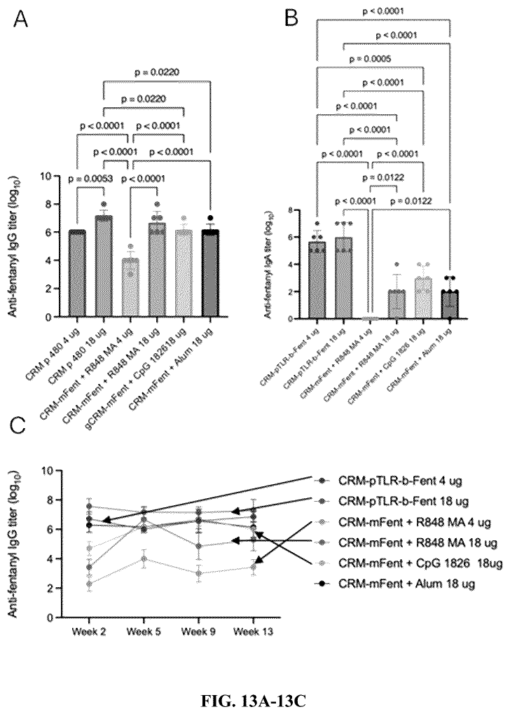

C57Bl/6 mice were vaccinated with CRM-pTLR-b-fent and CRM-mFent conjugate at two different doses 4 μg and 18 ug (of CRM adjuvanted with TLR 7/8 agonist, TLR 9 agonist CpG 1826, and alum weight equiv.) per mouse on day 0 and 21 (FIG. 13A). Mice were bled at different time points. Anti-fentanyl serum IgG (FIG. 13A) and IgA (FIG. 13B) antibody level determined by ELISA on week 5. Long term anti-fentanyl IgG responses were measured over 13 weeks (FIG. 13C). All statistical differences were determined by one-way ANOVA using Brown-Forsythe test where p<0.05 represent statistical significance with respect to all other treatment groups or as indicated. The end-point titers were defined as the highest dilution at which the 450 nm OD value was above the mean+4 SD of that of sample with no serum.

### Example 10

### Block Polymer Induces Better Protection

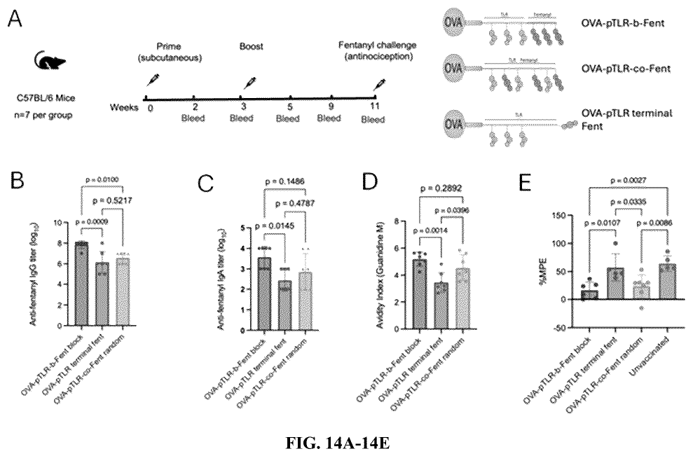

C57Bl/6 mice were vaccinated with OVA-pTLR-b-fent (block polymer), OVA-pTLR-co-fent (random polymer) and OVA-pTLR polymer with one terminal fentanyl at 10 μg of OVA and TLR weight equiv. per mouse on day 0 and 21 (FIG. 14A). Mice were bled at different time points. Anti-fentanyl serum IgG (FIG. 14B) and IgA (FIG. 14C) antibody level determined by ELISA on week 5. Avidity was measure using modified chaotropic ELISA using guanidine as denaturant. Avidity index (FIG. 14D) is a measurement of amount of guanidine required to inhibit the 50% binding of serum antibody on the plate. Nociception was measured on week 20 post vaccination using hot plate analgesia meter (Columbus instruments) post 30 min i.p. challenge of fentanyl HCl. (0.25 mg/kg). the baseline latency was measured prior to drug injection by observing one of the following nociception responses: hind paw licking or jumping with 60 s maximum cutoff time to prevent tissue damage. The percentage maximum possible effect (% MPE) (FIG. 14E) was calculated as the post-test latency minus the pretest latency divided by the maximum time (60 seconds) minus the pretest latency times 100. All statistical differences were determined by one-way ANOVA using Brown-Forsythe test where p<0.05 represent statistical significance with respect to all other treatment groups or as indicated. The end-point titers were defined as the highest dilution at which the 450 nm OD value was above the mean+4 SD of that of sample with no serum.

### Example 11

### Self-Immolative Linker Improve Antifentanyl Responses

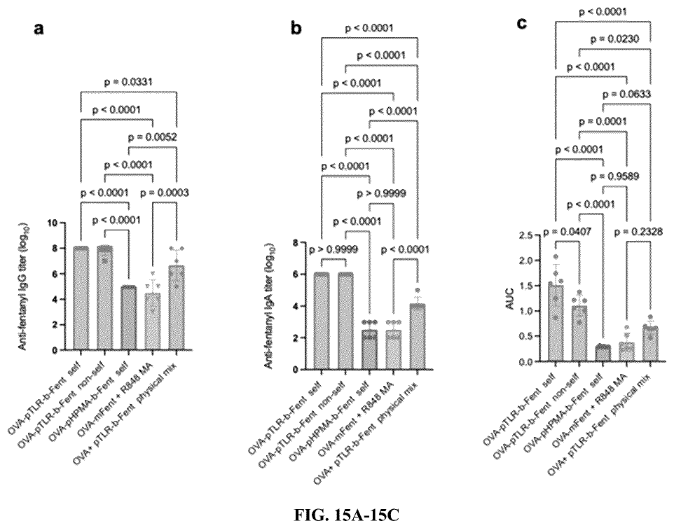

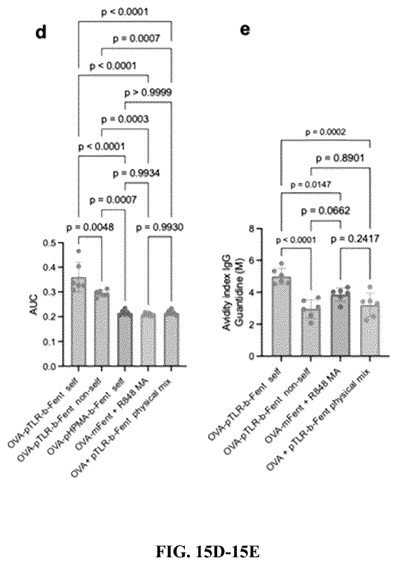

Polymeric vaccine where pTLR-b-Fent was conjugated to OVA by self immolative linker induces superior anti-fentanyl antibody responses than nonself linker conjugate vaccine. C57Bl/6 mice were vaccinated with OVA-pTLR-b-fent (with self-linker), OVA-pTLR-b-fent (non-self-linker), OVA-pHPMA-b-fent (with self-linker) and OVA-mFent conjugate at 10 ug OVA adjuvanted with admixed TLR 7/8 agonist or pTLR-b-Fent polymer weight equiv. per mouse on day 0 and 21. Mice were bled at different time points. Anti-fentanyl serum IgG (FIG. 15A) and IgA (FIG. 15B) antibody titer determined by ELISA on week 4. (FIG. 15A). Area under the curve for serum IgG (FIG. 15C) and IgA (FIG. 15D) was calculated using graph pad prism. Avidity was measure using modified chaotropic ELISA using guanidine as denaturant. Avidity index (FIG. 15E) is a measurement of amount of guanidine required to inhibit the 50% binding of serum antibody on the plate. All statistical differences were determined by one-way ANOVA using Brown-Forsythe test where p<0.05 represent statistical significance with respect to all other treatment groups or as indicated. The end-point titers were defined as the highest dilution at which the 450 nm OD value was above the mean+4 SD of that of naïve serum.

Although the invention has been described with reference to the presently preferred embodiment, it should be understood that various modifications can be made without departing from the spirit of the invention. Accordingly, the invention is limited only by the following claims.

### REFERENCES

ADDIN Mendeley Bibliography CSL_BIBLIOGRAPHY 1. Zoorob, M. Fentanyl shock: The changing geography of overdose in the United States. Int. J. Drug Policy 70, 40-46 (2019).
2. Sulima, A. et al. A Stable Heroin Analogue That Can Serve as a Vaccine Hapten to Induce Antibodies That Block the Effects of Heroin and Its Metabolites in Rodents and That Cross-React Immunologically with Related Drugs of Abuse. J. Med. Chem. 61, 329-343 (2018).
3. Kosten, T. & Owens, S. M. Immunotherapy for the treatment of drug abuse. Pharmacol. Ther. 108, 76-85 (2005).
4. Nutt, S. L., Hodgkin, P. D., Tarlinton, D. M. & Corcoran, L. M. The generation of antibody-secreting plasma cells. Nat. Rev. Immunol. 15, 160-171 (2015).
5. Slifka, M. K., Antia, R., Whitmire, J. K. & Ahmed, R. Humoral immunity due to long-lived plasma cells. Immunity 8, 363-372 (1998).
6. Dörner, T. & Radbruch, A. Selecting B cells and plasma cells to memory. J. Exp. Med. 201, 497-499 (2005).
7. Townsend, E. A. et al. Conjugate vaccine produces long-lasting attenuation of fentanyl vs. food choice and blocks expression of opioid withdrawal-induced increases in fentanyl choice in rats. Neuropsychopharmacology 44, 1681-1689 (2019).
8. Raleigh, M. D. et al. A fentanyl vaccine alters fentanyl distribution and protects against fentanyl-induced effects in mice and rats. J. Pharmacol. Exp. Ther. 368, 282-291 (2019).
9. Hwang, C. S. et al. Improved Admixture Vaccine of Fentanyl and Heroin Hapten Immunoconjugates: Antinociceptive Evaluation of Fentanyl-Contaminated Heroin. ACS Omega 3, 11537-11543 (2018).
10. Hwang, C. S. et al. Enhancing Efficacy and Stability of an Antiheroin Vaccine: Examination of Antinociception, Opioid Binding Profile, and Lethality. Mol. Pharm. 15, 1062-1072 (2018).
11. Raleigh, M. D., Pravetoni, M., Harris, A. C., Birnbaum, A. K. & Pentel, P. R. Selective effects of a morphine conjugate vaccine on heroin and metabolite distribution and heroin-induced behaviors in rats. J. Pharmacol. Exp. Ther. 344, 397-406 (2013).
12. Guo, J. et al. TLR4 agonist monophosphoryl lipid a alleviated radiation-induced intestinal injury. J. Immunol. Res. 2019, (2019).
13. Gefen, T. et al. The effect of haptens on protein-carrier immunogenicity. Immunology 144, 116-126 (2015).
14. Wilson, D. S. et al. Antigens reversibly conjugated to a polymeric glyco-adjuvant induce protective humoral and cellular immunity. Nat. Mater. 18, 175-185 (2019).
15. Castiblanco, D. P., Maul, R. W., Knode, L. M. R. & Gearhart, P. J. Co-stimulation of BCR and toll-like receptor 7 increases somatic hypermutation, Memory B cell formation, and secondary antibody response to protein antigen. Front. Immunol. 8, 1-9 (2017).
16. Wilson, D. S. et al. Synthetically glycosylated antigens induce antigen-specific tolerance and prevent the onset of diabetes. Nat. Biomed. Eng. 3, 817-829 (2019).
17. Wen, Y. et al. The regulators of BCR signaling during B cell activation. Blood Sci. 1, 119-129 (2019).
18. Finn, A. & Heath, P. Conjugate vaccines. Arch. Dis. Child. 90, 667-669 (2005).
19. Volkow, N. D., Collins, F. S. & Ph, D. Special Report The Role of Science in Addressing the Opioid Crisis. 391-394 (2020).
20. Nelson, J. C. et al. Compliance with multiple-dose vaccine schedules among older children, adolescents, and adults: Results from a vaccine safety datalink study. Am. J. Public Health 99, 389-397 (2009).
21. Palm, A. K. E. & Henry, C. Remembrance of Things Past: Long-Term B Cell Memory After Infection and Vaccination. Front. Immunol. 10, 1787 (2019).
22. McHeyzer-Williams, M., Okitsu, S., Wang, N. & McHeyzer-Williams, L. Molecular programming of B cell memory. Nat. Rev. Immunol. 12, 24-34 (2012).
23. Batista, F. D. & Harwood, N. E. The who, how and where of antigen presentation to B cells. Nat. Rev. Immunol. 9, 15-27 (2009).
24. Soni, C. et al. B Cell-Intrinsic TLR7 Signaling Is Essential for the Development of Spontaneous Germinal Centers. J. Immunol. 193, 4400-4414 (2014).
25. Cyster, J. G. & Allen, C. D. C. B Cell Responses: Cell Interaction Dynamics and Decisions. Cell 177, 524-540 (2019).
26. Hong, S. et al. B Cells Are the Dominant Antigen-Presenting Cells that Activate Naive CD4+ T Cells upon Immunization with a Virus-Derived Nanoparticle Antigen. Immunity 49, 695-708.e4 (2018).
27. Constant, S. L. B lymphocytes as antigen-presenting cells for CD4+ T cell priming in vivo. J. Immunol. 162, 5695-703 (1999).
28. Mogensen, T. H. Pathogen recognition and inflammatory signaling in innate immune defenses. Clin. Microbiol. Rev. 22, 240-273 (2009).
29. Bremer, P. T. et al. Combatting Synthetic Designer Opioids: A Conjugate Vaccine Ablates Lethal Doses of Fentanyl Class Drugs. Angew. Chemie-Int. Ed. 55, 3772-3775 (2016).
30. Pentel, P. R. et al. A nicotine conjugate vaccine reduces nicotine distribution to brain and attenuates its behavioral and cardiovascular effects in rats. Pharmacol. Biochem. Behav. 65, 191-198 (2000).
31. Ramakrishnan, M., Kinsey, B. M., Singh, R. A., Kosten, T. R. & Orson, F. M. Hapten optimization for cocaine vaccine with improved cocaine recognition. Chem. Biol. Drug Des. 84, 354-363 (2014).
32. Goniewicz, M. L. & Delijewski, M. Nicotine vaccines to treat tobacco dependence. Hum. Vaccines Immunother. 9, 13-25 (2013).
33. Petters, S., Westfall, J. M., Miller, B. F. Projected Deaths of Despair During the Coronavirus Recession, Well Being Trust. May 8, 2020. WellBeingTrust.org.
34. Hollingsworth, A., Ruhm, C. J., Simon, K. Macroeconomic conditions and opioid abuse. Journal of Health Economics 56, 222 (2017).
35. Dietze, P. et al. Effect of Intranasal vs Intramuscular Naloxone on Opioid Overdose: A Randomized Clinical Trial. JAMA Netw. open 2, e1914977 (2019).
36. Weiss, R. D. & Rao, V. The Prescription Opioid Addiction Treatment Study: What have we learned. Drug Alcohol Depend. 173, S48-S54 (2017).
37. Moad, G., Rizzardo, E. & Thang, S. H. RAFT polymerization and some of its applications. Chem.-An Asian J. 8, 1634-1644 (2013).
38. Hermann, C. D. et al. Rapidly polymerizing injectable click hydrogel therapy to delay bone growth in a murine re-synostosis model. Biomaterials 35, 9698-9708 (2014).
39. Garcia, A. J. et al. Self-assembling nanoparticles for intra-articular delivery of anti-inflammatory proteins. Biomaterials 33, 7665-7675 (2012).
40. Valdez, C. A., Leif, R. N. & Mayer, B. P. An efficient, optimized synthesis of fentanyl and related analogs. PLoS One 9, 2-9 (2014).
41. Yang, J., Chen, H., Vlahov, I. R., Cheng, J. X. & Low, P. S. Evaluation of disulfide reduction during receptor-mediated endocytosis by using FRET imaging. Proc. Natl. Acad. Sci. U.S.A 103, 13872-13877 (2006).
42. Shannack, M. M. & Linterman, M. A. T Follicular Helper Cells. Encycl. Cell Biol. 3, 541-549 (2016).

What is claimed is:

1. A conjugate comprising an immunogenic carrier protein coupled to a polymer, wherein the polymer comprises:
i) a first monomeric unit comprising a first monomer coupled to a hapten, and
ii) a second monomeric unit comprising a second monomer coupled to an immune cell receptor agonist.

2. The conjugate of claim 1, wherein the polymer further comprises a third monomeric unit comprising a third monomer.

3. The conjugate of claim 1, wherein the hapten is an opioid.

4. The conjugate of claim 3, wherein the opioid is fentanyl, a fentanyl analog, buprenorphine, codeine, dextromoramide, dihydrocodeine, enkephalin, heroin, hydrocodone, hydromorphone, meperidine, methadone, morphine, nicomorphine, opium, oxycodone, oxymorphone, pentazinine, pentazocine, methamphetamine, a derivative thereof, a precursor thereof, or pharmacologically acceptable salt or solvate thereof.

5. The conjugate of claim 4, wherein the opioid is fentanyl, a derivative thereof, a precursor thereof, or pharmacologically acceptable salt or solvate thereof.

6. The conjugate of claim 5, wherein the opioid is fentanyl.

7. The conjugate of claim 1, wherein the hapten is irreversibly coupled to the first monomer.

8. The conjugate of claim 1, wherein the immune cell receptor agonist is an agonist of an intracellular receptor.

9. The conjugate of claim 1, wherein the immune cell receptor agonist is a toll-like receptor agonist.

10. The conjugate of claim 1, wherein the TLR agonist is selected from the group consisting of a toll-like receptor 7 (TLR7) agonist, a toll-like receptor 8 (TLR8) agonist, and TLR7/TLR8 agonist.

11. The conjugate of claim 9, wherein the immune cell receptor agonist is a TLR7 agonist, selected from the group consisting of imiquimod, resiquimod, 852-A, vesatolimod, AZD8848, motolimod, selgantolimod, NKTR-262, RG-7854, DSP-0509, BDB-001, BDC-1001, LHC-165, SHR-2150, JNJ-4964, RO-711992, DN-1508052, VTX-1463, BNT-411, APR-003, ALT-702, GS-986, KUP-101, PRTX-007, PRX-034, S-34240, SBT-6050, SBT-6290, ZM-TLR8, VX-001, MBS-8, APR-002, and combinations thereof.

12. The conjugate of claim 9, wherein the TLR7 agonist is an imidazoquinoline.

13. The conjugate of claim 12, wherein the TLR7 agonist is

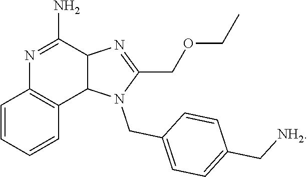 [SMILES: CCOCC1=NC2C(N)=Nc3ccccc3C2N1Cc1ccc(CN)cc1]

14. The conjugate of claim 1, wherein the immune cell receptor agonist is coupled to the second monomer by a cleavable linker.

15. The conjugate of claim 1, wherein the immunogenic carrier protein is a pathogenic protein, a bacterial protein, a tetanus protein, a tetanus toxin, a diphtheria toxin, a genetically detoxified diphtheria toxin, or CRM197 (CRM).

16. The conjugate of claim 1, wherein the immunogenic carrier protein coupled to the polymer by a cleavable linker.

17. A pharmaceutical composition comprising the conjugate of claim 1 and a pharmaceutically acceptable excipient.

18. A vaccine composition comprising a conjugate comprising an immunogenic carrier protein coupled to a polymer, wherein the polymer comprises:
i) a first monomeric unit comprising a first monomer coupled to a hapten,
ii) a second monomeric unit comprising a second monomer coupled to an immune cell receptor agonist, and
iii) a third monomeric unit comprising a third monomer.

19. The vaccine composition of claim 18, wherein the immunogenic carrier protein is ovalbumin, the hapten is fentanyl and the immune cell receptor agonist is a TLR7 agonist, a TLR8 agonist, or a TLR7/TLR8 agonist.

20. The vaccine composition of claim 18, wherein the immunogenic carrier protein is CRM197, the hapten is fentanyl, and the immune cell receptor agonist is a TLR7 agonist, a TLR8 agonist, or a TLR7/TLR8 agonist.

21. The vaccine composition of claim 17, wherein the immunogenic carrier protein is Keyhole Limpet Hemocyanin (KLH), the hapten is fentanyl, and the immune cell receptor agonist is a TLR7 agonist, a TLR8 agonist, or a TLR7/TLR8 agonist.

22. A method of preventing drug overdose in a subject in need thereof comprising administering to the subject a conjugate comprising an immunogenic carrier protein coupled to a polymer, wherein the polymer comprises:
i) a first monomeric unit comprising a first monomer coupled to a hapten, and
ii) a second monomeric unit comprising a second monomer coupled to an immune cell receptor agonist,
thereby preventing drug overdose in the subject.

23. The method of claim 22, wherein the immunogenic carrier protein is ovalbumin, the hapten is fentanyl, and the immune cell receptor agonist is a TLR7 agonist, a TLR8 agonist, or a TLR7/TLR8 agonist.

24. The method of claim 22, wherein the immunogenic carrier protein is CRM197, the hapten is fentanyl, and the immune cell receptor agonist is a TLR7 agonist, a TLR8 agonist, or a TLR7/TLR8 agonist.

25. The method of claim 20, wherein the immunogenic carrier protein is Keyhole Limpet Hemocyanin (KLH), the hapten is fentanyl, and the immune cell receptor agonist is a TLR7 agonist, a TLR8 agonist, or a TRL7/TLR8 agonist.
